(19) United States
(12) Patent Application Publication
(10) Pub. No.: US 2013/0096054 A1
(43) Pub. Date: Apr. 18, 2013

(54) LATENT HUMAN IMMUNODEFICIENCY VIRUS REACTIVIATION

(71) Applicants: Olaf Kutsch, Birmingham, AL (US); Michael Niederweis, Homewood, AL (US); Frank Wolschendorf, Birmingham, AL (US); Alexandra Duverger, Birmingham, AL (US); Frederic Wagner, Birmingham, AL (US)

(72) Inventors: Olaf Kutsch, Birmingham, AL (US); Michael Niederweis, Homewood, AL (US); Frank Wolschendorf, Birmingham, AL (US); Alexandra Duverger, Birmingham, AL (US); Frederic Wagner, Birmingham, AL (US)

(73) Assignee: THE UAB RESEARCH FOUNDATION, Birmingham, AL (US)

(21) Appl. No.: 13/698,490

(22) PCT Filed: May 18, 2011
(86) PCT No.: PCT/US11/36992

§ 371 Date: Dec. 27, 2012

Related U.S. Application Data
(60) Provisional application No. 61/345,924, filed on May 18, 2010.

(51) Int. Cl. C07K 14/16; A61K 45/00; A61K 38/16

(52) U.S. Cl. CPC C07K 14/16; A61K 38/162; A61K 45/00
USPC 514/4.1; 435/375; 435/252.1; 435/69.1; 530/350

(57) ABSTRACT

Provided herein are methods or reactivating a latent Human Immunodeficiency Virus (HIV) infection in a cell. The methods comprise modulating a level of NF-κB activity in the cell by contacting the cell with an agent that produces a transient first increase in the level of NF-κB activity without a second delayed increase in NF-κB activity. Optionally, a second agent is used to prime the reactivation. Also provided herein is an isolated Massilia bacterium or population thereof capable of producing a HIV-1 reactivating factor (HRF). Also provided are methods of culturing the Massilia bacteria. Further provided are methods of reactivating a latent Human Immunodeficiency Virus-1 (HIV-1) infection in a subject comprising administering to the subject a HIV-1 reactivating factor produced by Massilia bacteria, optionally with a priming agent.

### CROSS REFERENCE TO RELATED APPLICATION

This application claims the benefit of U.S. Provisional Application No. 61/345,924, filed on May 18, 2010, which is incorporated by reference herein in its entirety.

### STATEMENT REGARDING FEDERALLY FUNDED RESEARCH

This invention was made with government funding under Grant Nos. AI077457 and AI064012 from the National Institutes of Health. The government has certain rights in this invention.

### BACKGROUND

Highly active antiretroviral therapy (HAART) quickly suppresses HIV-1 replication in patients to non-detectable levels. Even after years of effective HAART regimen, however, cessation of therapy results in the immediate rebound of viremia. This is attributed to a long-lived reservoir of latently HIV-1 infected memory CD4+ T cells. As a result of the long lifespan of memory T cells that serve as cellular hosts to latent HIV-1 infection, the latent HIV-1 reservoir is extremely stable. Natural eradication, in the absence of any replenishment of the reservoir by de novo infection events, is predicted to take about 70 years. As natural depletion of the latent reservoir is unlikely to be achievable, HIV-1 latency is believed to represent the principal obstacle to curative AIDS therapy.

### SUMMARY

Provided herein are methods of reactivating a latent Human Immunodeficiency Virus (HIV) infection in a cell. The methods comprise modulating a level of NF-κB activity in the cell by contacting the cell with a first agent that produces a transient first increase in the level of NF-κB activity without a second delayed increase in NF-κB activity. Optionally, the methods comprise contacting the cell with a second agent (e.g., actinomycin D, aclacinomycin or amphotericin B). The second agent primes the latent HIV infection in the cell. Optionally, the second agent reduces the dosage required for reactivation of the latent HIV infection by the first agent.

Also provided are methods of reactivating a latent HIV infection in a subject by administering to the subject an HIV reactivating factor (HRF) or a reactivating fragment of a HRF produced by Massilia bacteria.

Also provided is an isolated Massilia bacterium or population thereof that is for producing an HRF.

Further provided are methods of producing an HRF. The methods comprise culturing a Massilia bacterium in a mammalian cell culture medium.

### DESCRIPTION OF DRAWINGS

FIGS. 1A and 1B show culture supernatants from Massilia timonae reactivate latent HIV-1 infection. FIG. 1A shows the results of flow cytometric analysis of latently HIV-1 infected CA5 reporter T cells treated with 10 μl of Massilia timonae cell culture supernatant (HRF). Untreated control cells and HRF-treated cells were subjected to flow cytometric analysis to determine enhanced green fluorescent protein (EGFP) expression as a direct and quantitative marker of HIV-1 expression 24 hours post stimulation. FSC/SSC dot plots are represented to evaluate cell viability as a function of changes in the FSC (cell size)—SSC (granularity) phenotype of the cells. FIG. 1B is a histogram showing the reproducibility of the observed HIV-1 reactivation. Three independent preparations of HRF (batches HRF1-3) on four different latently infected T cell lines (5F3, 8E12, CA5 and 12D4) were tested. The percentage of EGFP positive cells following stimulation is depicted in the histogram. Cell culture supernatants of Pseudomonas aeruginosa cultures, grown under similar conditions, were used as specificity controls.

FIGS. 2A-2C show Massilia timonae mediates reactivation of latent HIV-1 infection. FIG. 2A shows the 16S rRNA sequence (SEQ ID NO:1) of the identified and cloned bacteria is >99.8% identical to Massilia timonae 16S rRNA sequence. R represents either an A or a T. FIG. 2B is an image showing Massilia timonae colonies grown on blood agar. FIG. 2C is an image of a gel and a graph showing the HIV-1 reactivating capabilities of different Massilia timonae strains. Massilia timonae strain #701 and #703 were purchased from the ATCC and grown under identical conditions and cell density as the isolated Massilia timonae comprising HRF. Supernatants were harvested, normalized for cell density and loaded on a SDS-page gel. Protein concentration and distribution was visualized using a silverstain method (left panel). The HIV-1 reactivating capacity of the three supernatants was determined by titrating sterile-filtered bacterial supernatants on latently HIV-1 infected CA5 T cells and then quantifying the level of HIV-1 reactivation in the cell population by determining the percentage of EGFP-positive cells in the total cell population (right panel).

FIGS. 3A-3C show the characterization of HRF properties. FIG. 3A is a graph demonstrating that the HIV-1 reactivating capacity of HRF was diminished when Massilia timonae culture preparations were exposed to increasing concentrations of trypsin or proteinase K. Trypsin and proteinase K were used to test whether the HIV-1 reactivating capacity was due to a bacterial protein. The treated supernatants were transferred on CA5 T cells and the capacity to reactivate latent HIV-1 infection was determined by flow cytometric analysis for EGFP expression. FIG. 3B is a histogram demonstrating that the HIV-1 reactivating capacity of HRF was unaffected by DNase or RNase. Similar experiments were performed using DNAse (5U and 20 U) and RNAse (2 μg and 10 μg) to treat culture supernatants from Massilia timonae cultures to determine whether the HIV-1 reactivating capacity is related to the presence of bacterial DNA or RNA molecules. FIG. 3C is a histogram demonstrating the size of the HRF in the Massilia timonae culture supernatant. Massilia timonae culture preparations were subjected to size-exclusion filtration using filters with the indicated kDa cut-offs. Flow through and supernatant for each preparation were transferred to CA5 T cells and the HIV-1 reactivating capacity was determined by flow cytometric analysis for EGFP-positive cells.

FIGS. 4A and 4B show HRF-mediated reactivation is not the result of pyrogenic activity. As Massilia timonae is a gram-negative bacterium, it was tested whether HIV-1 reactivation could be triggered by endotoxin-like activities. FIG. 4A is a graph demonstrating the HIV-1 reactivating capacity of HRF preparations after the removal of endotoxins. To remove endotoxins from the HRF preparations, the Massilia timonae culture supernatants were incubated with polymyxin B-agarose prior to stimulation of the latently HIV-1 infected CA5 reporter T cells with increasing doses of the HRF preparations. Levels of HIV-1 reactivation were determined as the percentage of EGFP-positive cells using flow cytometric analysis 48 hours post stimulation. FIG. 4B is a graph demonstrating the HIV-1 reactivating capacity of increasing concentrations of HRF and LPS on the latently HIV-1 infected CA5 reporter T cells and the monocytic reporter cell line (THP89GRP cells). The latently HIV-1 infected CA5 reporter T cells were treated with increasing concentrations of HRF or LPS. Functionality of LPS was demonstrated by stimulating the latently HIV-1 infected monocytic THP89GFP cells with increasing amounts of LPS. For all conditions, levels of HIV-1 reactivation were determined as the percentage of EGFP-positive cells 48 hours post stimulation using flow cytometric analysis.

FIG. 5 shows HIV-1 reactivation triggered by co-culture of Massilia timonae and CA5 T cells. Mixtures of Massilia timonae and constant numbers of CA5 T cells (1×10⁶ cells) at different ratios were co-cultured for 24 hours. Using a logarithmic representation of the FSC/SSC analysis, it was possible to visualize Massilia timonae and the T cells in the same FSC/SSC dot plot using flow cytometric analysis. By gating on the T cell population, the level of HIV-1 reactivation as a function of the number of bacteria was determined. The dot plots represent the experimental conditions with the maximum number of bacteria tested (upper quadrants) and the lowest number of bacteria that still provide full reactivation (lower quadrants). FSC/SSC plots were used to determine any changes in cell morphology and viability. EGFP was determined as a direct marker of HIV-1 expression.

FIGS. 6A-6C show HRF triggers suboptimal functional induction of NF-κB dependent gene expression. FIG. 6A is a graph demonstrating HIV-1 reactivating kinetics of HRF relative to known HIV-1 reactivating agents that signal through the NF-κB pathway. The HIV-1 reactivating kinetics were determined by stimulating CA5 T cells with optimal concentrations of HRF, phorbol 12-myristate 13-acetate (PMA) and tumor necrosis factor-α (TNF-α) and analyzing HIV-1 reactivation as a function of EGFP-expression over a period of 48 hours. Data for all agents are depicted as the percentage of EGFP-positive cells in the total cell population. FIG. 6B is a graph demonstrating the capacity of HRF for TAT-independent activation of the HIV-1 LTR. To test the ability of HRF to mediate Tat-independent activation of the HIV-1 LTR, NOMI cells (HIV-1 reporter cell line with an integrated LTR-EGFP construct) were stimulated with increasing concentrations of PMA (0.01-10 ng/ml), TNF-α (0.003-30 μg/ml) and HRF supernatant (0.3-100 μl). The level of LTR-induction was then measured as EGFP-expression detectable after 24 hours as determined by flow cytometric analysis. Arrows indicate the concentrations at which the respective stimulus would have triggered full HIV-1 reactivation in the latently HIV-1 infected CA5 T cell line. FIG. 6C shows histograms demonstrating the effect of HRF on NF-κB dependent promoters in 293T cells. 293T cells were transfected with several NF-κB dependent promoter constructs and then stimulated with either TNF-α (10 μg/ml) or HRF (25 μl). Promoter induction was measured as total EGFP expression. (NF-κB:pNF-κB-d2EGFP; LTR:HIV-1 LTR-GFP; IL-8:pIL-8 GFP; TNF:human TNF-GFP; MSCV:murine stem cell virus LTR driven GFP; no NF-κB responsive elements).

FIGS. 7A-7D are graphs showing the kinetics of HRF-mediated induction of NF-κB activity. Jurkat or latently HIV-1 infected CA5 T cells were stimulated with optimal concentrations of PMA and HRF, and cells were harvested at the indicated time points. Nuclear extracts were generated and (FIGS. 7A and 7C)NF-κB p50 activity and (FIGS. 7B and 7D) NF-κB p65 activity were determined using the TransAM™ NF-κB family ELISA kit (Active Motif; Carlsbad, Calif.).

FIGS. 8A-8C show HRF induced NF-κB and cytokine expression in peripheral blood mononuclear cells (PBMCs). FIG. 8A are graphs demonstrating the kinetics of NF-κB stimulation by HRF and PHA-L in PBMCs. PBMCs were activated with optimal concentrations of either PHA-L or with HRF. Cells were harvested at the indicated time points. Nuclear extracts were generated and NF-κB p50 activity and NF-κB p65 activity were determined using the TransAM™ NF-κB family ELISA kit (Active Motif). FIG. 8B is a histogram demonstrating that HIV-1 replication in PBMCs in the presence of saturating HRF levels was not significantly increased as compared to a control. PBMCs from four different healthy donors were stimulated with an anti-CD3/CD28 antibody combination and infected with a GFP reporter virus on day 4 post-stimulation. HIV-1 replication was monitored in the absence (C) or presence of HRF for 5 days and the achieved HIV-1 infection levels were determined by flow cytometric analysis for the percentage of EGFP-positive cells. All infections were normalized to the infection level in the untreated culture and the histogram represents the mean infection levels obtained in 4 donor cultures±standard deviation. FIG. 8C are graphs demonstrating that HRF did not induce meaningful levels of pro-inflammatory cytokine secretion. PBMCs from four healthy donors were left unstimulated (C) or stimulated with an anti-CD3/CD28 antibody combination as a positive control or were stimulated with a concentration of HRF that would trigger maximum HIV-1 reactivation in CA5 T cells. Culture supernatants were collected after 24 hours and the concentrations of a panel of cytokines was determined by BioPlex analysis. Cytokine concentrations for TNF-α (top panel), IFN-γ (middle panel), and IL-8 (bottom panel) are presented for four individual donors.

FIGS. 9A-C show flow cytometry-based high throughput screen (HTS) for HIV-1 reactivating drug combinations. FIG. 9A shows time resolved acquisition of cell samples in a 96-well plate-based assay format. Each peak represents the accumulated events of one well. FIG. 9B shows that automated peak recognition allows backgating of the fluorescent barcode (RFP) to quantify on-target effects (GFP) in the three populations treated with different activators (J89GFP: HRF; J89GFP-R: anti-CD3 mAb OKT3; J89GFP-R+: PMA). FIG. 9C shows the results of high throughput flow cytometric analysis in a 96 well plate using a HyperCyt® autosampler (Intellicyt Corporation; Albuquerque, N. Mex.). The level of induced reactivation by each compound in combination with sub-optimal concentrations of OKT3 (0.1 μg/ml; white), HRF (gray) and PMA (0.3 ng/ml; black) is plotted as percent reactivation over the well number and is shown on the left. The symbols in the figure on the right represent the corresponding viability of each sample. Note that only the total viability of each well was plotted and not for each of the individual RFP-barcoded populations.

FIGS. 10A-10B shows dactinomycin (actinomycin D) primes latent HIV-1 infection for reactivation in a time dependent manner. FIG. 10A shows flow cytometric analysis of latently HIV-1 infected J89GFP T cells left unstimulated (C) or treated for 18 hours with dactinomycin (DM). Then either population was stimulated with a sub-optimal concentration of TNF-α. Reactivation levels were determined by measuring the level of GFP-positive cells 24 hours post TNF-α addition using flow cytometric analysis. FIG. 10B shows flow cytometric analysis of J89GFP T cells treated for varying amounts of time with dactinomycin and then stimulated with TNF-α. Levels of reactivation were quantified by measuring the level of GFP expressing cells.

FIGS. 11A-D show optimal concentration of dactinomycin for HIV-1 reactivation and comparison with other transcription inhibitors or DNA intercalators. CA5 T cells were pretreated with increasing concentrations of dactinomycin (DM) (FIG. 11A), the DNA intercalator daunorubicin (DR) (FIG. 11B), or the transcription inhibitors DRB (FIG. 11 C) and α-amanitin (FIG. 11D) (concentrations indicated). The cells were then left unstimulated (white circles) or activated with a sub-optimal concentration of HRF (black circles). Reactivation levels were determined as the percentage of GFP-positive cells 24 hours after stimulation. Viability of the cells correlates with the size of the symbol (size range adjusted to 5-95%).

FIGS. 12A-F show the effect of dactinomycin on active HIV-1 infection. Optimal HIV-1 reactivating agents should not boost active HIV-1 infection to minimize the risk of de novo infections. To test the effect of dactinomycin on active HIV-1 transcription the drug was titrated on chronically active infected GFP-reporter T cells JNLG#35 (black) and JNLG#44 (white). The results for dactinomycin were compared with those for other drugs/compounds that would exert similar reported mechanisms of action than dactinomycin. Changes in HIV-1 transcription levels were determined after 48 hours by measuring GFP mean channel fluorescence intensity (GFP MCF). GFP MCF was plotted over the drug concentration for dactinomycin (FIG. 12A), the DNA intercalators daunorubicin (DM) (FIG. 12B) and rebeccamycin (RM) (FIG. 12C), as well as the transcription inhibitors ICRF-193 (FIG. 12D), DRB (FIG. 12E) and α-amanitin (FIG. 12F). The size of symbols indicates the respective cell viability in the sample (size range adjusted to 5-95%).

FIGS. 13A-B show the effect of dactinomycin on latent infection as a function of the orientation of integration relative to the direction of host-gene transcription. HIV-1 can integrate in the same transcriptional orientation as the host-gene or in the converse sense. Two latently HIV-1 infected T cell lines, which were determined to have HIV-1 integrated in the same sense orientation (CA5 cells) (FIG. 13A) and in the converse sense orientation (EF7 cells) (FIG. 13B) were used. For either cell line, the host-gene name, the position of integration and the chromosome number is given. For either T cell line, reactivation levels achieved by TNF-α treatment alone or by TNF-α stimulation following pretreatment with 4 ng/ml dactinomycin for 18 hours are shown. Reactivation levels were determined using flow cytometric analysis for GFP expression. The gray dotted line represents maximum achievable reactivation levels using PMA.

FIG. 14 shows that dactinomycin primes latent HIV-2 infection for reactivation. J2574 reporter T cells were infected with HIV-2 7312A and a latently infected cell population was established (>90% latently infected cells). The cell population was then pretreated with varying concentrations of dactinomycin (0-8 ng/ml) for 18 hours and then either left untreated (C) or stimulated with a sub-optimal dose of HRF, which by itself triggered reactivation in 10% of the cells. Reactivation levels were determined 24 hours post HRF activation by quantifying the level of GFP-positive cells using flow cytometric analysis.

FIGS. 15A-C show that HMBA primes latent HIV-1 infection for reactivation. FIG. 15A shows a FACS analysis demonstrating that HMBA triggers HIV-1 reactivation in the utilized reporter cells lines, and that HMBA can prime HIV-1 for reactivation. Latently HIV-1 infected CA5 T cells were treated with an optimal dose of HMBA (27% reactivation), a sub-optimal dose of TNF-α (32% reactivation, or a combination of HMBA and TNF-α (81% reactivation). FIG. 15B shows a graph demonstrating the optimal dose of HMBA. Increasing amounts of HMBA were titrated on latently HIV-1 infected CA5 T cells. The optimal concentration of HMBA as a HIV-1 priming agent, prior to the onset of drug mediated toxicities was between 3-10 mM. FIG. 15C shows a graph demonstrating that HMBA HMBA can primer for HIV-1 reactivation with multiple agents. Latently HIV-1 infected CA5 T cells were treated with increasing doses of HMBA alone, HMBA plus TNF-α, HMBA plus PMA, or HMBA plus HRF.

FIGS. 16A-D show dactinomycin releases P-TEFb from its inactive complex with HEXIM-1. To test the ability of dactinomycin to act as priming agent for HIV-1 reactivation by releasing P-TEFb from its inactive complex with HEXIM-1, glycerol gradient analysis was performed to determine the effect of dactinomycin on the P-TEFb-HEXIM-1 complex composition in the latently infected J89GFP T cells. J89GFP cells were left untreated, treated with a high concentration of dactinomycin for 1 hour or with the physiological optimal concentration (0.004 μg/ml or 0.01 μg/ml) for 18 hours. Cell lysates were separated on a glycerol gradient (10-45%). Each gradient fraction was separated on a 10% SDS-PAGE gel and transferred by Western blot. FIG. 16A shows a quantitative analysis of the band intensity of the Western blots stained with an anti-CDK9 antibody (FIG. 16C) to reveal drug induced shifts in the complex composition. Band densities were determined using ImageJ and are presented as relative band density. FIG. 16B shows band density analysis of the same Western blot experiments performed using anti-HEXIM-1 antibody (FIG. 16D). Band densities were determined using ImageJ and are presented as relative band density.

FIG. 17 shows two drugs/compounds that prime latent HIV infection for reactivation by HIV reactivating factor (HRF). Latently HIV-1 infected CA5 cells were pretreated for 20 hours with increasing concentrations of the indicated drugs/compounds. ACM: aclacinomycin; ActD: actinomycin D had a strong priming effect; the RNAP II inhibitor DRB: 5,6-dichloro-1-β-D-ribobenz-imidazole; Dauno: daunorubicin; MG132: a proteasome inhibitor; and the RNAP II inhibitor α-aminitin, which are reported to exert similar inhibitory effects, either as DNA intercalators or as transcription inhibitors did not exhibit any priming effect on HIV-1 reactivation and demonstrated the specificity of the effect observed following application of aclacinomycin or dactinomycin. The cells were then stimulated with a sub-optimal dose of HRF and the effects of HIV-1 reactivation were determined by flow cytometric analysis for the percentage of GFP-positive cells.

### DETAILED DESCRIPTION

Antiretroviral therapy (ART) can suppress, but not eradicate, HIV-1 infection, as the virus can integrate itself in a dormant or latent state into the genome of long-lived immune cells. The integrated virus persists indefinitely and spreads if therapy is halted. It is believed that the most promising way to eradicate latent HIV-1 infection is to reactivate these viruses. Infected cells with reactivated virus would become susceptible to destruction by the immune system or would be destroyed by viral cytotoxicity, thereby deleting this source of residual virus. Unfortunately, stimuli that reactivate latent HIV-1 infection can cause a deadly “cytokine storm,” the equivalent of an anaphylactic shock. The methods provided herein, however, reactivate a latent Human Immunodeficiency Virus (HIV) without producing a deadly cytokine storm.

Further, previous drug screens for HIV-1 reactivating compounds or previous attempts to therapeutically reactivate latent HIV-1 infection in patients were developed under the “one-drug one-target” hypothesis, which is based on the premise that the perfect chemical probe acts on a single target. However, research on the molecular mechanisms controlling HIV-1 latentcy indicates that multiple components should be triggered in coordinated fashion to induce HIV-1 reactivation in the absence of sustained T cell activation. This takes into consideration that all genes function in the context of other genes or that molecular control mechanisms function in the context of a network and that there really cannot be a single target, as biological systems respond dynamically and variably based on the activities of interacting genes or mechanisms. Thus, the methods provided herein optionally use combinations of drugs to reactivate latent HIV infections.

Provided herein is a novel Human Immunodeficiency Virus (HIV) reactivating factor (HRF) and compositions comprising the novel HRF. Such compositions include culture media comprising HRF produced by Massilia bacterium. Also provided herein are nucleic acid sequences capable of encoding an HRF. Optionally, the HRF is produced from a Massilia bacterium. Optionally, the HRF is produced by a Massilia timonae strain deposited on May 18, 2010 in accordance with the Budapest Treaty with the ATCC, 10801 University Road, Manassas, Va. 20110, with the strain designation HRF having ATCC Accession number PTA-10969. Optionally the HRF is produced by Massilia timonae strain having ATCC accession number BAA-703. Optionally, the HRF modulates a level of NF-κB activity. Optionally, the HRF comprises a polypeptide greater than or equal to 50 kilodaltons (kDa). Optionally, the HRF comprises a polypeptide less than or equal to 100 kDa.

The HRFs provided herein show little to no cytotoxicity and have a therapeutic index greater than 300. A therapeutic index is a comparison of the amount of a therapeutic agent that causes a therapeutic effect to the amount that causes death. The therapeutic index is a ratio given by the lethal dose of a drug or agent for 50% of the population (LD₅₀) divided by the minimum effective therapeutic dose for 50% of the population (ED₅₀). A high therapeutic index is preferable.

Modulating the level of NF-κB activity in the cell by contacting the cell with a first agent results in a transient first increase in the level of NF-κB activity without a delayed second increase in NF-κB activity. Thus, the transient first increase in the level of NF-κB activity is not followed by a sustained level of NF-κB activity. A sustained level of NF-κB activity, can, for example, result in the induction of cytokine gene expression and a concomitant delayed increase. As described herein, the first agent produces a transient first increase in the level of NF-κB activity, resulting in a peak level of NF-κB activity, with the level of NF-κB subsequently decreasing over time. Little or no second peak of activity occurs.

The delayed second increase in NF-κB activity may be associated with cytokine gene induction. The absence or reduction of a delayed second increase in NF-κB activity results in the absence of substantial cytokine gene induction. Optionally, the absence of cytokine gene induction comprises the absence of substantial induction of one or more of TNF-α, IL-8, IFNγ, IL-2, IL-4, and IL-6. By substantial cytokine gene induction is meant an increase over control that is significantly different than control values using standard statistical analysis.

The modulation of NF-κB activity differs in pattern from a modulation caused by TNF-α, PMA, PHA-L, IL-2, anti-CD3 monoclonal antibodies, or a combination of anti-CD-3 and anti-CD28 monoclonal antibodies. The modulation of NF-κB activity caused by TNF-α, PMA, PHA-L, IL-2, anti-CD3 monoclonal antibodies, or a combination of anti-CD-3 and anti-CD28 monoclonal antibodies can, for example, produce a pattern of NF-κB activity. Optionally, the pattern of NF-κB activity caused by these agents begins with a first increase in the level of NF-κB activity, followed by a sustained increased level of NF-κB activity. The sustained level of NF-κB activity can, for example, be an oscillating level of NF-κB activity. An oscillating pattern of NF-κB activity includes an increase in level of NF-κB activity, a decrease in level of NF-κB activity, and another increase, but the pattern can continue to repeat.

Optionally, the latent HIV infection is primed in the cell by administration of a second agent. The second agent primes latent HIV-1 infection for reactivation by lowering the activation threshold for latent infection. Full reactivation can then be triggered by a reactivating factor, which by itself at a low dose would have little or no effect on latent infection, and most importantly, would not trigger or would trigger minimal cytokine expression or any other detrimental side effects. By way of an example, administration of the second agent can reduce the amount (i.e., dosage) of the first agent needed to reactivate the latent HIV infection in the cell.

The second agent can be administered to the subject prior to or concomitantly with the first agent. The second agent can, for example, prime the latent HIV infection by releasing P-TEFb from an inactive complex comprising HEXIM-1 and 7SK RNA. Optionally, the second agent is selected from the group consisting of actinomycin D, aclacinomycin, ampotericin B, and WP631.

The second agent, can, for example, prime the latent HIV infection to be reactivated in a manner not limited to HRF. By way of an example, priming the latent HIV infection with actinomycin D, aclacinomycin, amphotericin B, or WP631 can allow for suboptimal doses of other agents, including for example, TNF-α, IL-2, or CD3 antibody, to reactivate the latent HIV infection. Without intending to be limited in theory, priming the latent HIV infection affects the modulation of NF-κB activity by the suboptimal dose of TNF-a, IL-2, or CD3, which avoids triggering a “cytokine storm.”

Also provided are compositions comprising a purified population of Massilia bacteria. Massilia timonae is a gram-negative bacterium, which was initially isolated from a severely immuno-compromised human patient in the context of an opportunistic infection. Massilia timonae is considered non-pathogenic and frequently appears in soil samples, drinking water, air, and even in a spacecraft assembly clean room. Optionally, the purified population comprises a Massilia timonae strain having ATCC Accession number PTA-10969. Optionally, the composition comprises Massilia timonae strain having ATCC accession number BAA-703. The Massilia strains can, for example, produce a HIV reactivating factor (HRF). Also provided are compositions comprising the HRFs produced by the Massilia stains provided herein.

Further provided are isolated Massilia bacteria or populations thereof. The isolated Massilia bacteria or populations thereof are capable of producing a Human Immunodeficiency Virus (HIV) reactivating factor (HRF). Optionally, the Massilia bacteria comprise a 16S rRNA sequence, wherein the 16S rRNA sequence comprises at least 95% sequence identity with the 16S rRNA sequence of Massilia timonae. Optionally, the 16S rRNA sequence comprises at least 99% sequence identity with the 16S rRNA sequence of Massilia timonae.

The similarity of sequence identity or sequence similarity between two nucleic acid sequences can be obtained, for example, by the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989, which are herein incorporated by reference for at least material related to nucleic acid alignment.

Provided herein are compositions containing HRF polypeptides, nucleic acids encoding HRFs, Massilia bacterial strains capable of producing HRFs, optionally with one or more priming agents, anti-retroviral agents, and a pharmaceutically acceptable carrier described herein. The herein provided compositions are suitable for administration in vitro or in vivo. By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. The carrier is selected to minimize degradation of the active ingredient and to minimize adverse side effects in the subject.

Suitable carriers and their formulations are described in Remington: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the priming agent, reactivating agent and/or anti-retroviral agent, e.g., the small molecule, polypeptide, nucleic acid molecule, and/or peptidomimetic, to humans or other subjects.

The compositions are administered in a number of ways depending on whether local or systemic treatment is desired, and on the area to be treated. The compositions are administered via any of several routes of administration, including topically, orally, parenterally, intravenously, intra-articulately, intraperitoneally, intramuscularly, subcutaneously, intracavity, transdermally, intrahepatically, intracranially, nebulization/inhalation, or by installation via bronchoscopy.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable.

Optionally, a nucleic acid molecule or polypeptide is administered by a vector comprising the nucleic acid molecule or a nucleic acid sequence encoding the polypeptide (e.g., a nucleic acid sequence encoding the HRF produced by the Massilia strains provided herein). There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based deliver systems. Such methods are well known in the art and readily adaptable for use with the compositions and methods described herein.

Also provided are methods of producing a HIV reactivating factor (HRF). The methods comprise culturing Massilia bacteria in a mammalian cell culture medium under conditions that allow for the secretion of the HRF into the culture media and isolating the Massilia bacteria conditioned media. Optionally, the Massilia bacteria comprises a Massilia timonae strain having ATCC Accession number PTA-10969. Optionally, the Massilia bacteria comprises a Massilia timonae strain having ATCC accession number BAA-703. Optionally, the mammalian cell culture medium comprises a RPMI 1640 medium. Optionally, the RPMI 1640 medium further comprises a mammalian serum, bovine serum albumin (BSA), or myoglobin. Optionally, the RPMI medium can comprise about 1 to about 20% of mammalian serum, BSA, or myoglobin. The mammalian serum can, for example, be fetal bovine serum (FBS). The RPMI 1640 medium can comprise about 5% to about 15% FBS. Optionally, the RPMI 1640 medium comprises about 10% FBS. Optionally, the RPMI 1640 medium further comprises bovine serum albumin (BSA). The RPMI 1640 medium can, for example, comprise about 0.1 to about 20 mg per ml of BSA. Optionally, the RPMI 1640 medium further comprises myoglobin. The myoglobin can, for example, be obtained from a horse, a pig, a cow, a human, or from any other primate. Optionally, the HRF is isolated from mammalian culture medium. Isolation of the HRF from the mammalian culture medium is performed using methods known in the art, e.g., see Woolley and Al-Rubeai, Biotechnol. Bioeng. 104(3):590-600 (2009); Kalyanpur, Mol. Biotechnol. 22:87-96 (2002); Sanchez et al., FEMS Microbiol. Lett. 295(2):226-9 (2009); Dowling et al., Anticancer Res. 27(3A):1309-17 (2007) and as taught herein regarding fractionation of the medium.

Also provided herein are methods of reactivating a latent Human Immunodeficiency Virus (HIV) infection in a cell. The methods comprise modulating a level of NF-κB activity in the cell by contacting the cell with a first agent that produces a transient first increase in the level of NF-κB activity without a delayed second increase in NF-κB activity. The modulation in the level of NF-κB activity can, for example, be detected as a modulation in the level of NF-κB p50 or NF-κB p65 activity. The modulation in the level of NF-κB activity does not result in the induction of HIV replication. Optionally, the cell is in vitro or in vivo.

Optionally, the methods comprise contacting the cell with a second agent that primes the latent HIV infection. The second agent can, for example, releasing P-TEFb from a complex. The complex can comprise HEXIM-1 and 7SK RNA. Optionally, the second agent is selected from the group consisting of actinomycin D, aclacinomycin, amphotericin B, and WP631.

Also provided are methods of reactivating a latent Human Immunodeficiency Virus (HIV) infection in a subject. The methods comprise administering to the subject an HIV reactivating factor (HRF) produced by Massilia bacteria or a reactivating fragment of the HRF produced by Massilia bacteria. Optionally, the HRF is administered to the subject by directly administering the Massilia bacteria or Massilia conditioned medium or a fraction thereof to the subject. Optionally, the HRF is administered to the subject as a bacterial supernatant isolated from cultured Massilia bacteria. The bacterial supernatant can be isolated from the cultured Massilia bacteria by methods known in the art and as described herein. Optionally, the methods comprise administering to the subject an agent that primes the latent HIV infection in the subject. By priming the latent HIV infection, it is meant that the agent modulates or alters the latent HIV infection to allow for a more efficient reactivation of the HIV infection by the HRF. By way of an example, administration of the agent can reduce the amount (i.e., dosage) of the HRF needed to reactivate the latent HIV infection in the subject. Optionally, the agent is administered prior to or concomitant with the administration of the HRF. Optionally, the second agent is selected from the group consisting of actinomycin D, aclacinomycin, amphotericin B, and WP631.

Actinomycin D, amphotericin B or aclacinomycin is administered prior to or simultaneously with the reactivating agent. Optionally, actinomycin D is administered about 6-30 hours (e.g., 12-24 hours) prior to administration of the reactivating agent. Amphotericin B or aclacinomycin can, for example, be administered up to 12 hours (e.g., about 6 hours) prior to or simultaneously with the reactivating agent.

Actinomycin D, for example, is administered at a dose of up to about 15 micrograms per kilogram per day (μg/kg/day). Optionally, actinomycin D can be administered at a range of about 400-600 milligrams per meter squared body area per day (mg/m²/day). Actinomycin D can be administered at this range for 1-5 days; however, treatment can be stopped and restarted after a five day dosing period. Aclacinomycin is administered at a dosage of up to about 100 mg/m²/day for a maximum of five days. Amphotericin B, for example, is administered at a dose of about 1.5 mg/kg/day. Optionally, amphotericin B is administered at a dose of 0.1 mg/ml.

Also provided are methods of treating an HIV infection in a subject. The methods comprise administering to the subject a first agent that reactivates a latent HIV infection by modulating a level of NF-κB activity, wherein modulation of the level of NF-κB activity comprises producing a transient first increase in the level of NF-κB activity without a second delayed increase in NF-κB activity; and administering to the subject an anti-retroviral agent. Administration of the anti-retroviral agent results in the treatment of the HIV infection. Optionally, the anti-retroviral agent is administered to the subject after reactivation of the latent HIV infection or concomitantly with the first agent. Optionally, the subject is administered a second agent that primes the latent HIV infection in the subject. The second agent can be administered to the subject prior to or concomitantly with the first agent. The second agent can, for example, prime the latent HIV infection by releasing P-TEFb from an inactive complex of HEXIM-1 and 7SK RNA. Optionally, the second agent is selected from the group consisting of actinomycin D, aclacinomycin, amphotericin B, and WP631.

The anti-retroviral agent can, for example, be selected from the group consisting of a nucleoside, a nucleoside reverse transcriptase inhibitor (NRTI), a non-nucleoside reverse transcriptase inhibitor (NNRTI), a nucleoside analog reverse transcriptase inhibitor (NARTI), a protease inhibitor, an integrase inhibitor, an entry inhibitor, a maturation inhibitor, and combinations thereof.

Any of the aforementioned second agents or therapeutic agents (e.g., actinomycin D or an anti-retroviral agent) can be used in any combination with the compositions described herein. Combinations are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to concomitant, simultaneous, or sequential administration of two or more agents.

As used herein, the terms peptide, polypeptide, or protein are used broadly to mean two or more amino acids linked by a peptide bond. Protein, peptide, and polypeptide are also used herein interchangeably to refer to amino acid sequences. It should be recognized that the term polypeptide is not used herein to suggest a particular size or number of amino acids comprising the molecule and that a peptide of the invention can contain up to several amino acid residues or more.

The methods and agents as described herein are useful for therapeutic treatment. Therapeutic treatment involves administering to a subject a therapeutically effective amount of the agents described herein after diagnosis of HIV infection. The terms effective amount and effective dosage are used interchangeably. The term effective amount is defined as any amount necessary to produce a desired physiologic response (e.g., an effective amount of a reactivating agent reactivates a latent HIV infection in at least about 50% of the total cell population; an effective amount of a priming agent primes a latent HIV infection by reducing the effective amount of the reactivating agent needed to reactive a latent HIV infection; and an effective amount of an anti-retroviral agent results in a reduction in HIV viral load 30-100 fold within six weeks with the viral load falling below detectable limits within 4-6 months). Effective amounts and schedules for administering the agent may be determined empirically, and making such determinations is within the skill in the art. The dosage ranges for administration are those large enough to produce the desired effect (e.g., HIV reactivation and/or reduction of HIV symptoms). The dosage should not be so large as to cause substantial adverse side effects, such as unwanted cross-reactions, anaphylactic reactions, and the like. Dosages of HRF can, for example, be reduced with a prime dosage of a second agent such as actinomycin D, aclacinomycin, amphotericin B, and WP631. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

As used herein the terms treatment, treat, or treating refers to a method of reducing or delaying the effects of a disease or condition (e.g., HIV infection) or symptom of the disease or condition (e.g., treatment results in an increase in CD4⁺ T cells and a reduction in HIV viral load). Thus in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Example 1

### Reactivation of Latent HIV-1 Infection without Cytokine Gene Induction

### Materials and Methods

Cell Culture and Reagents.

All T cell lines, as well as the latently HIV-1 infected monocytic THP89GFP cells were maintained in RPMI 1640 supplemented with 2 mM L-glutamine, 100 U/ml penicillin, 100 μg/ml streptomycin and 10% heat inactivated fetal bovine serum. Fetal bovine serum was obtained from HyClone (Logan, Utah) and was tested on a panel of latently infected cells to assure that it did not spontaneously trigger HIV-1 reactivation (Jones et al., Assay Drug Dev. Technol. 5:181-9 (2007); Kutsch et al., J. Virol. 76:8776-86 (2002)). The phorbol ester 13-phorbol-12-myristate acetate (PMA), LPS and polymixin B-agarose were purchased from Sigma (St. Louis, Mo.), whereas recombinant human TNF-α was obtained from R&D Systems (Minneapolis, Minn.).

The utilized EGFP reporter virus HIV-1 NLENG1-IRES has been described elsewhere (Kutsch et al., J. Virol. 76:8776-86 (2002); Levy et al., Proc. Natl. Acad. Sci. USA 101:4204-9 (2004)). The reporter plasmid pNF-κB-d2EGFP was purchased from Clontech (Mountain View, Calif.). The LTR-GFP construct and the IL-8 reporter construct have been described earlier (Choi et al., Mol. Cell. Biol. 22:724-36 (2002)). The TNF-promoter construct was generated by cloning the human TNF-α promoter element defined by primer pair 5′-BglII; 5′-GGCGCGGAGATCTTAACGAAGACAGGGCCA TGT-3′ (SEQ ID NO:2) and 3′-AgeI; 5′-GCCAATACCGGTGTGTCCTTTCCAGGG GAGAG-3′ (SEQ ID NO:3) into pd2EGFP (Clontech). MSCV-GFP was generated by cloning the EGFP-gene into retroviral pMSCV-puro vector (Clontech).

Flow Cytometry.

Infection levels in the cell cultures were monitored by flow cytometric analysis of EGFP expression. Flow cytometric analysis was performed on a GUAVA EasyCyte (Millipore; Billerica, Mass.), or a LSRII (Becton & Dickinson; Franklin Lakes, N.J.).

BioPlex Analysis.

Following stimulation of the peripheral blood mononuclear cells (PBMCs) with PHA-L or HIV-1 reactivating factor (HRF), supernatant samples were collected at time points between 12 and 48 hours post stimulation. Preliminary analysis revealed that peak cytokine secretion was seen around 24 hours. Therefore, cytokine levels in culture supernatant samples from all donors were determined at the 24 hour time point using a customized Milliplex mAP kit for the simultaneous analysis of six human cytokines (IL-2, IL-4, IL-6, IL-8, TNF-α and IFN-γ). BioPlex analysis was performed on a Luminex 100 (BioRad; Hercules, Calif.).

Preparation of Cytoplasmic and Nuclear Protein Extracts.

Cells were grown in RPMI medium supplemented with 10% FBS and 1% PSG to approximately 5×10⁵ cells per milliliter. Cells were centrifuged and resuspended in fresh pre-warmed medium. PMA, HRF and/or JNKiV were added immediately at the indicated concentrations. The final cell density for the assay was 1×10⁶ cells per milliliter. The culture flasks were kept in a humidified CO₂ incubator at 37° C. For each time point, a 1 ml cell suspension was removed and immediately centrifuged at full speed in a tabletop centrifuge for 30 seconds. The cell pellet was washed in 1 ml ice cold PBS, quickly centrifuged again and frozen at −80° C. To prepare total protein extracts, the frozen cell pellets were resuspended in ice-cold RIPA buffer (Cell Signaling Technology, Danvers, Mass.) and incubated at 4° C. for 40 minutes. Samples were vortexed every 10 minutes during that time. After centrifugation at 16000 g for 10 minutes, the protein containing supernatant was carefully removed. To obtain cytoplasmic and nuclear protein extracts the NE-PER nuclear and cytoplasmic protein reagents (ThermoFisher; Waltham, Mass.) were used according to the manufacturer's instructions. The protein concentration of the extracts was determined by using the BCA protein assay Kit (ThermoFisher). Briefly, 2 μl of total and cytoplasmic proteins and 5 μl of nuclear protein extracts were mixed with water to give a final volume of 25 ul in a 96 well plate. To this, 200 μl of the dye reagent, which was mixed and prepared according to the manufacturer's protocol, were added and incubated for 30 minutes to 1 hour at 37° C. The absorbance at 595 nm was determined using a 96 well plate reader (Synergy HT, BIO-Tek; Winooski, Vt.).

Direct Quantification of Relative NF-κB Activity.

NF-κB activity in nuclear extracts was quantified using the TransAM™ NF-κB family ELISA kit from Active Motif, Inc. (Carlsbad, Calif.) according to the manufacturer's instructions.

Bacterial Isolation and Identification.

Following several rounds of cloning on blood agar plates, bacterial chromosomal DNA from single clones was isolated using phenol extraction. Briefly, cells were pelleted by centrifugation, resuspended in Chloroform:Methanol (3:1) and vortexed. The same volume of TRIS-buffered phenol/chloroform/isoamyl alcohol was added and the mixture was vortexed before the addition of GTC buffer. After mixing, the sample was vortexed and centrifuged at 9000 g for 20 minutes. The upper phase was carefully removed and DNA was precipitated by isopropanol, washed with 70% ethanol, dried in an vacuum centrifuge for 15 minutes and resuspended in 100 μl purified water. The 16S rRNA gene was amplified using the primer pair (16SrRNA for: 5′-AGAGTTTGATCCTGGCTCAG-3′ (SEQ ID NO:4); 16SrRNArev: 5′-ACGGCTACCTTGTTACGACTT-3′ (SEQ ID NO:5)). These and the following primers were used to sequence the 16S rRNA (Mass16sF#2 5′-CCCTAAACGATGTCTACTAGTTGT-3′ (SEQ ID NO:6); MassRNA5 5′-TTCGGGCACAACCAAATCTCTTCG-3′ (SEQ ID NO:7); and MassRNA4 5′-GGCTCAACCTCCCAATTGCGATG-3′ (SEQ ID NO:8)). Based on the 16S rRNA sequence the bacterium was identified as Massilia timonae (NIH BLAST). Except for 3 nucleotides, the 16S rRNA gene of the isolated HRF producing Massilia strain was identical to the sequence of Massilia timonae (ATCC# BAA-701) and (ATCC# BAA-703), which correspond to gene sequence AY157759 and AY157761, respectively (La Scola et al., J. Clin. Microbiol. 36:2847-52 (1998)).

Bacterial Growth.

Bacteria from various sources were isolated on TSA II agar plates containing 5% sheep blood. Bacterial isolates were then grown in RPMI 1640 medium supplemented with 10% FBS. After two days of incubation at 37° C. bacteria were pelleted and resuspended in RPMI medium to an optical density (OD600) of 8, aliquoted and frozen at −80° C. Unless otherwise indicated, Massilia timonae was grown in RPMI 1640 medium supplemented with 10% FBS. Typically, medium was inoculated with Massilia timonae from frozen stocks to an OD600 of 0.01. After 48 hours at 37° C., the culture was centrifuged at 3200 g to separate bacteria from the culture medium. Bacteria were resuspended in fresh medium to an OD600 of 8 and frozen to be used as references and stock cultures. The supernatant was then centrifuged at 10,000 g for 20 minutes and sterilized by passage through an 0.2 μm PVDF filter with low protein binding ability. HRF activity was determined by its ability to reactivate latent HIV-1 infection in CA5 cells. Only supernatants of which 6 μl reactivated infection in at least 60% of CA5 cells were used to study HRF properties.

HRF Characterization.

To determine the chemical nature of HRF, 100 μl of HRF containing culture filtrate were treated with different amounts of Proteinase K, Trypsin, DNAse or RNAse for 15 minutes at 37° C. The enzymes were inactivated at 95° C. for 5 minutes. HRF was concentrated by ammonium sulfate precipitation using standard protocols. Briefly, the best concentration for HRF precipitation was determined by adding ammonium sulfate to a final concentration of 20%, 40%, 60% or 80% (w/v). After 14 hours at 4° C., the precipitated proteins were retrieved by centrifugation for 40 minutes at 16,000 g. The pellet was reconstituted in PBS buffer. Ammonium sulfate from supernatants and precipitated proteins was removed by passage through a 3 kDa molecular weight cut off membrane (Microcon, Millipore) according to the manufacturer's recommendations. Fresh, ice cold PBS was added when 75% of the sample volume had passed through the filter. This procedure was repeated four times. Latently HIV-1 infected CA5 T cells tolerate ammonium sulfate up to 5% (w/v) in the cell culture medium without any sings of HIV-1 reactivation. As a result of the molecular weight cutoff (MWCO) filtration procedure, the highest possible ammonium sulfate concentration in cell culture was 0.3% as the filtration and washing results in a 256-fold dilution. Proteins were precipitated by 60% w/v ammonium sulfate from 10 ml bacterial culture filtrate. The pellet was resuspended in 250 ml PBS. Ammonium sulfate was removed by MWCO filtration as described above. Protein concentration was determined using the BCA protein assay Kit (ThermoFisher) according to the manufacturer's recommendations. 1.5 μg of protein were then separated on a 10% polyacrylamide gel according to standard protocols. Separated proteins were visualized by silver staining

### Results

### Identification of a Bacterium Secreting a Novel HIV-1 Reactivating Protein.

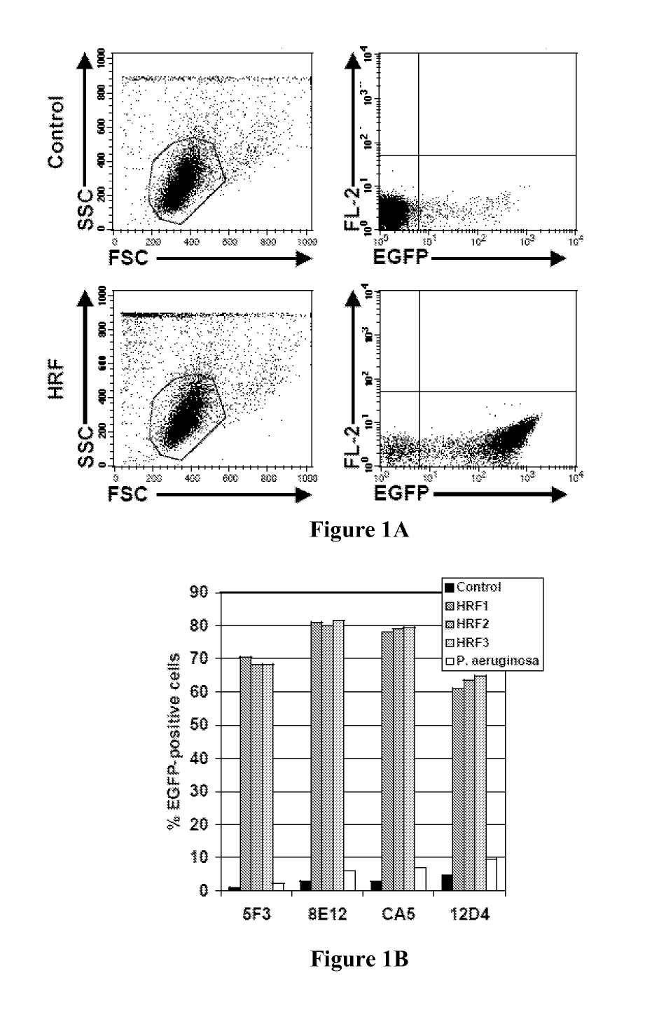

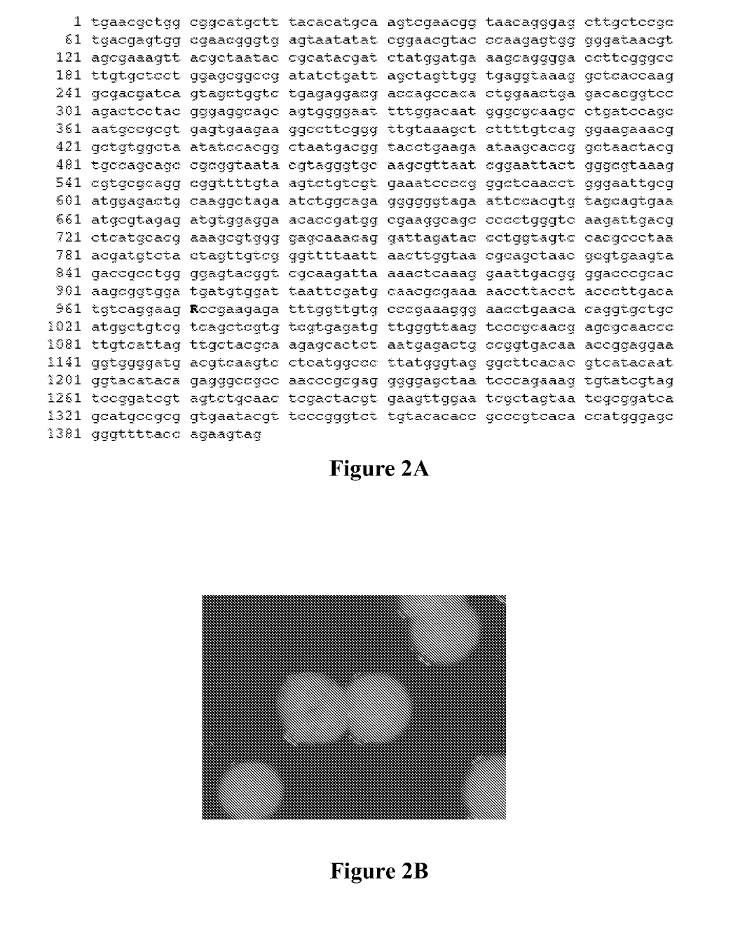

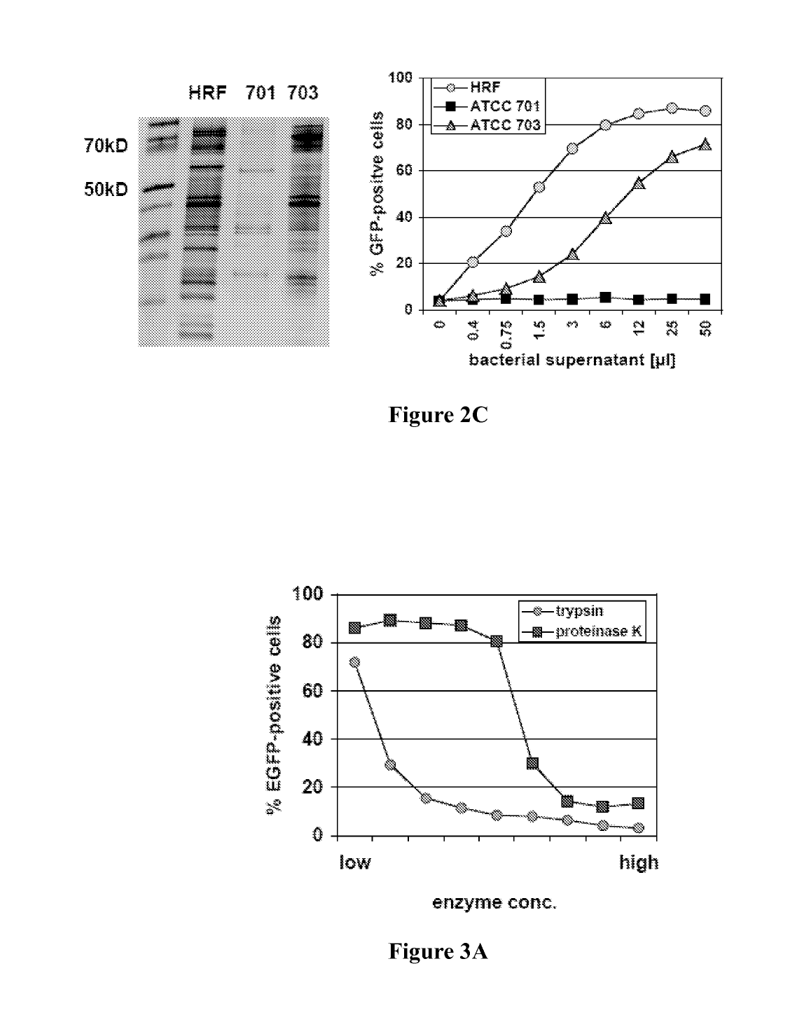

A potent HIV-1 reactivating activity in the cell culture supernatant filtrate of an initially unknown bacterium was identified. Upon addition of this culture supernatant to latently HIV-1 infected reporter T cell lines in which GFP expression serves as a direct and quantitative marker of HIV-1 expression (Duverger et al., J. Virol. 83:3078-93 (2009)), high levels of HIV-1 reactivation were observed. No effect on cell viability as seen by flow cytometric FSC/SSC analysis was observed (FIG. 1A). The bacteria were cloned and isolated on blood agar plates and the 16S rRNA identified the bacterium as Massilia timonae (>99% sequence homology over 1,400 base pairs (bp); FIG. 2A). The isolated Massilia timonae strain was deposited on May 18, 2010 with the ATCC with the strain designation HRF. This strain was designated ATCC Accession number PTA-10969. The isolated bacterium did not grow in Luria-Bertani (LB), Hartman DeBond (HdB) or Brain Heart infusion medium. The latter is the recommended growth medium for Massilia timonae. Several of the Massilia timonae strains deposited at the ATCC either produced no, or much lower HIV-1 reactivating capacity, which correlated with the overall diminished activity of these strains to secrete proteins into the culture supernatant (FIG. 2C).

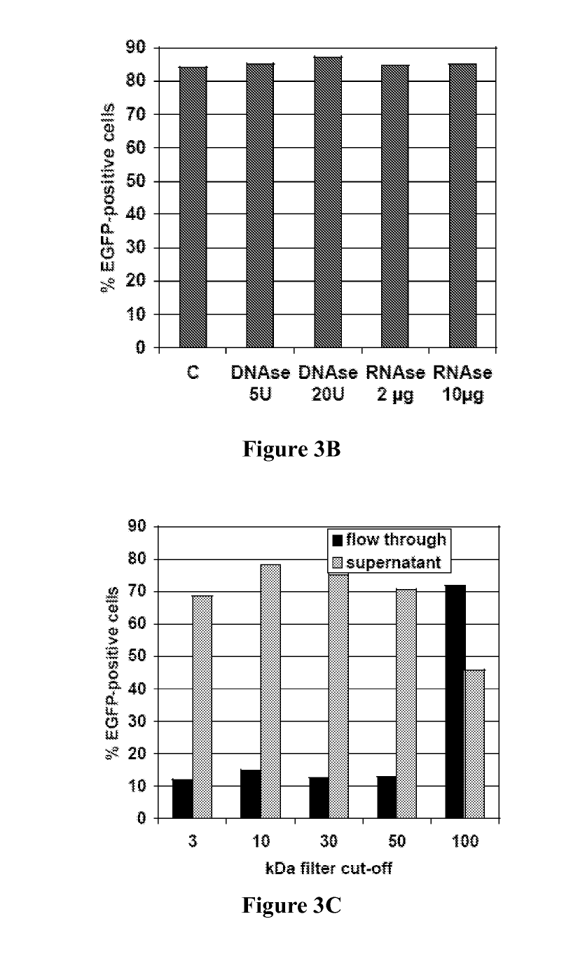

HRF activity could be removed from the supernatants by chloroform and acetonitrile precipitation. HRF activity would precipitate in >40% ammonium sulfate solutions and could be fully reconstituted in watery solutions. HRF activity was sensitive to trypsin and proteinase K digestion (FIG. 3A). Treatment of the supernatants with DNase or RNase did not impair the HIV-1 reactivating ability of HRF (FIG. 3B). Taken together these data suggest that HRF is a polypeptide, which as determined by size exclusion HPLC and molecular weight size exclusion filtration has a molecular weight in the range of 50-100 kD (FIG. 3C).

Initial Characterization of HRF Effect on Latent HIV-1 Infection.

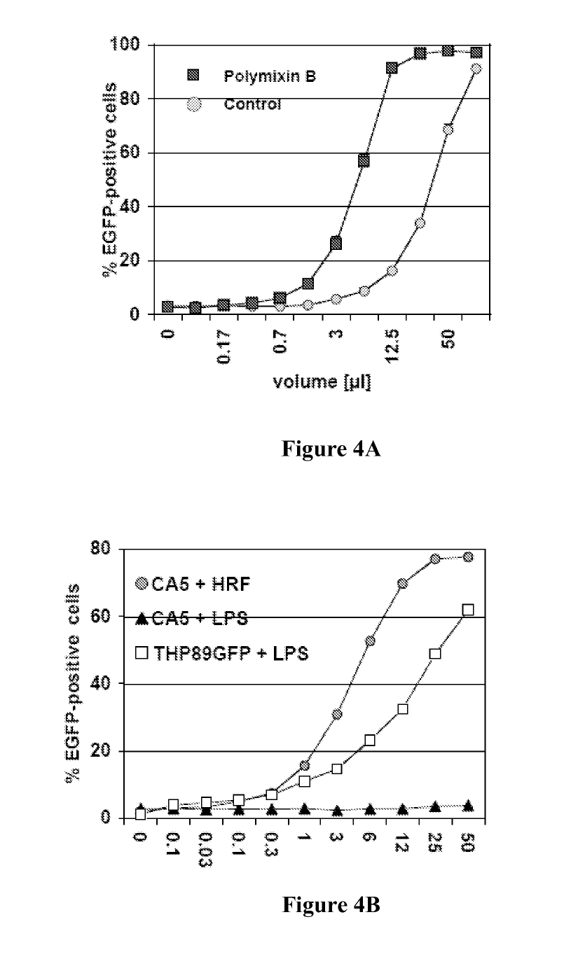

HRF was found to efficiently reactivate latent HIV-1 infection in the four tested latently infected reporter T cell lines developed previously (FIG. 1B) (Duverger et al., J. Virol. 83:3078-93 (2009); Jones et al., Assay Drug Dev. Technol. 5:181-9 (2007); Kutsch et al., J. Virol. 76:8776-86 (2002)). Culture supernatants from other gram-negative bacteria, e.g., from Pseudomonas aeruginosa or E. coli cultures, had no reactivating effect on these cell lines. Reactivation occurred in a concentration dependent manner and was not triggered by any endotoxin contamination, as was demonstrated by treatment of the bacterial culture filtrate with polymyxin B or endotoxin removal on polymyxin B-agarose columns (FIG. 4A). Also, while HRF reactivated latent HIV-1 infection in latently HIV-1 infected reporter T cell lines and monocytic reporter cell lines (THP89GFP) (Kutsch et al., J. Virol. 76:8776-86 (2002)), lipopolysaccharide, a prototypic endotoxin, only reactivated HIV-1 infection in the latently HIV-1 infected monocytic THP89GFP cells (FIG. 4B).

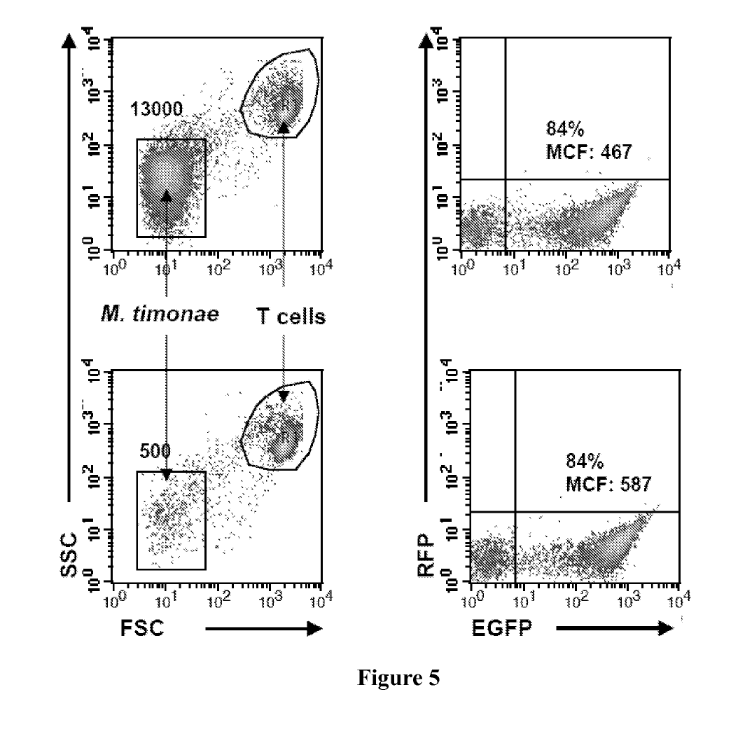

The isolated Massilia timonae strain, in contrast to other bacteria (e.g., Pseudomonas aeruginosa), did not overgrow the T cell cultures and was usually eliminated by the cells. In co-culture, as few as 500 bacteria triggered HIV-1 reactivation in a population of 1×10⁶ latently infected T cells, whereas a 25-fold excess of bacteria still did not impair viability of the T cell culture (FIG. 5).

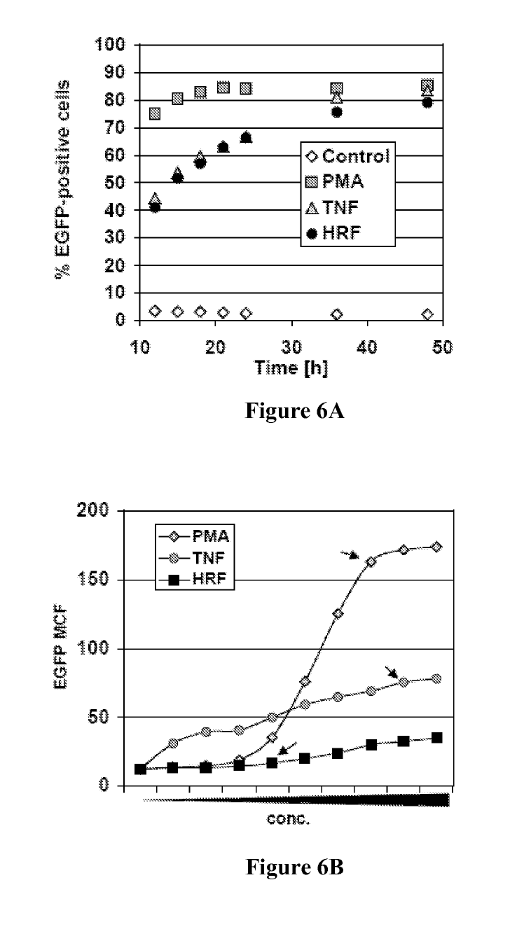

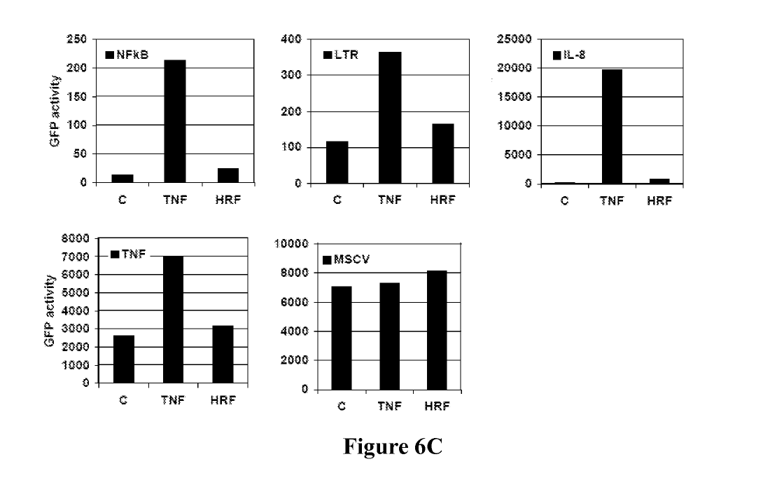

HRF triggers NF-κB activity spike. HRF activated latent HIV-1 provirus with a potency comparable to that of TNFα or PMA. Reactivation kinetics were similar to those of TNF-α and less rapid then reactivation kinetics seen following stimulation with PMA (FIG. 6A). However, in contrast to these non-therapeutic agents, HRF was far less cytotoxic in cell culture and showed a therapeutic index of >300.

As the NF-κB pathway has been recognized as vital to HIV-1 activation, the ability of HRF to trigger NF-κB activation was investigated. It was initially determined that HRF had the ability to stimulate Tat independent activation of an integrated LTR-GFP construct in NOMI reporter T cells. In these cells, both TNF-α and PMA stimulated Tat-independent activation of the integrated LTR-GFP construct, at concentrations that correlated with those concentrations required to trigger efficient HIV-1 reactivation in latently infected cells. In NOMI cells, HRF produced a modest increase in GFP expression, which was only observed at HRF concentrations that exceeded the HRF concentration required to trigger HIV-1 reactivation in latently infected T cells (FIG. 6B). In addition, HRF induced weak GFP expression driven by constructs that harbor the NF-κB responsive IL-8- or TNF-α-promoters, a HIV-1 LTR-GFP construct and a GFP construct under the control of three consensus NF-κB sites transfected into 293T cells (Choi et al., Mol. Cell. Biol. 22:724-36 (2002); Ochsenbauer-Jambor et al., Biotechniques 40:91-100 (2006)). As a control, all promoters were efficiently induced by TNF-α. Neither TNF-α nor HRF influenced GFP expression of a construct under the control of the murine stem cell virus LTR, which lacks a NF-κB responsive element (FIG. 6C).

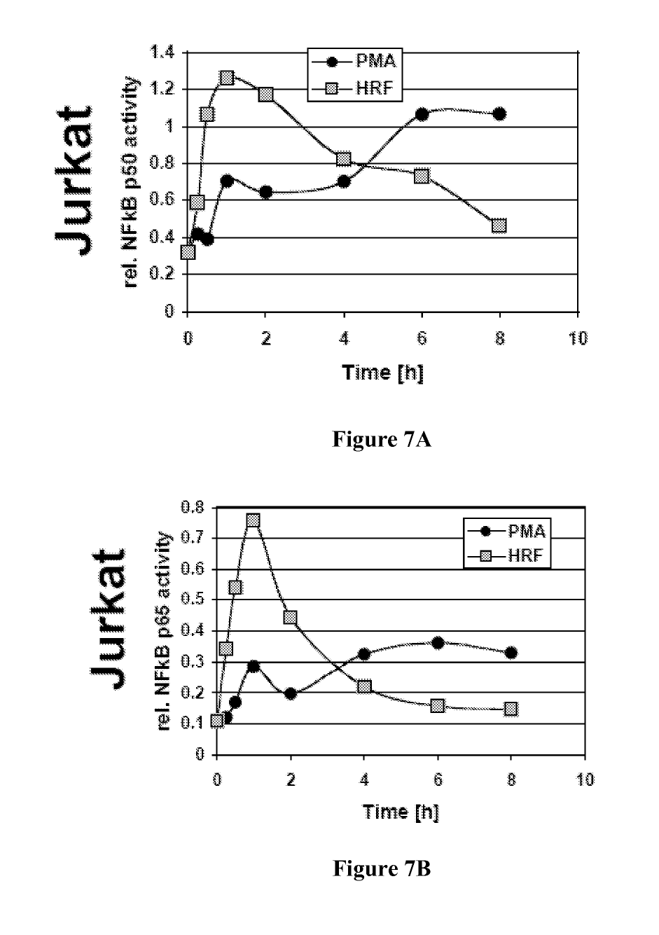

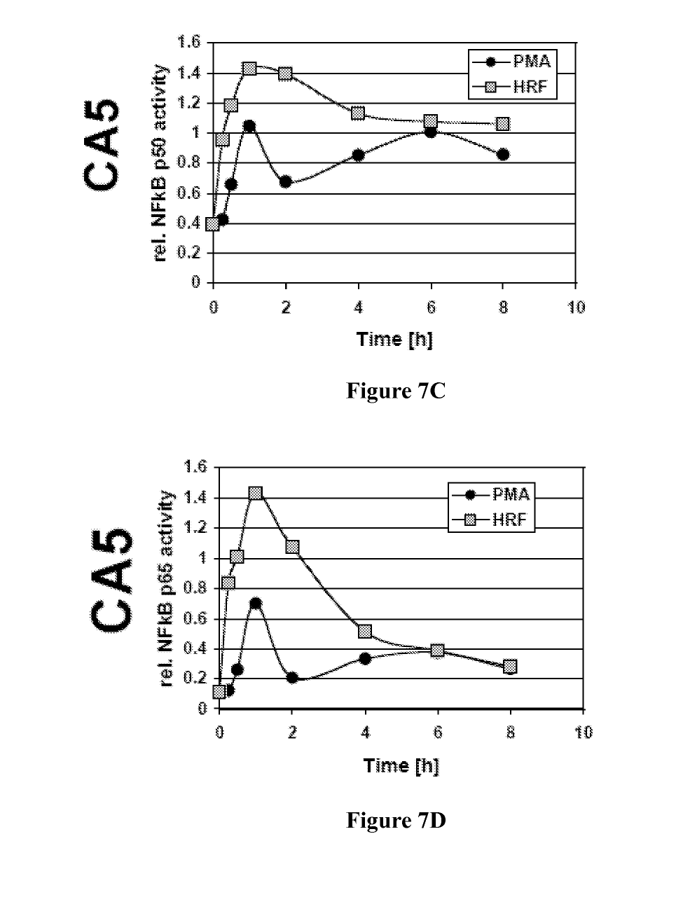

In contrast to these functional results, it was found that at the molecular level, HRF potently and with fast kinetics induced NF-κB p50 and p65 activation. HRF-mediated NF-κB activation in Jurkat cells had a ˜3-fold increased amplitude for p50 and ˜3-fold increased amplitude for p65 when compared to PMA stimulation. However, HRF-induced NF-κB activity in the parental Jurkat T cells was less sustained in comparison to PMA-stimulated NF-κB activation (FIG. 7). Lack of sustained NF-κB activation could explain why cellular promoters were only induced weakly and why HRF induced a low level of Tat-independent HIV-1 LTR activity (FIG. 6B). However, such low level LTR activity would be sufficient to initiate Tat expression. Initial low-level Tat production then initiates full Tat transactivation and thereby promotes the self-perpetuating increase to full HIV-1 expression. As Tat has been reported to stimulate NF-κB activity, this should also lead to sustained high NF-κB activity, which has earlier been defined as a prerequisite for efficient HIV-1 reactivation (Williams et al., J. Virol. 81:6043-56 (2007)).

Indeed, the kinetic NF-κB activity profile in the latent CA5 T cells following HRF stimulation is identical to that seen in the parental Jurkat cells for the first 4 hours, after which the HRF induced NF-κB p50 activity stabilizes at an elevated level, suggesting the onset of a second activating mechanism, likely Tat protein expression.

HRF Stimulates NF-κB in PBMCs, but HRF Fails to Promote Relevant Levels of Cytokine Induction in PBMCs.

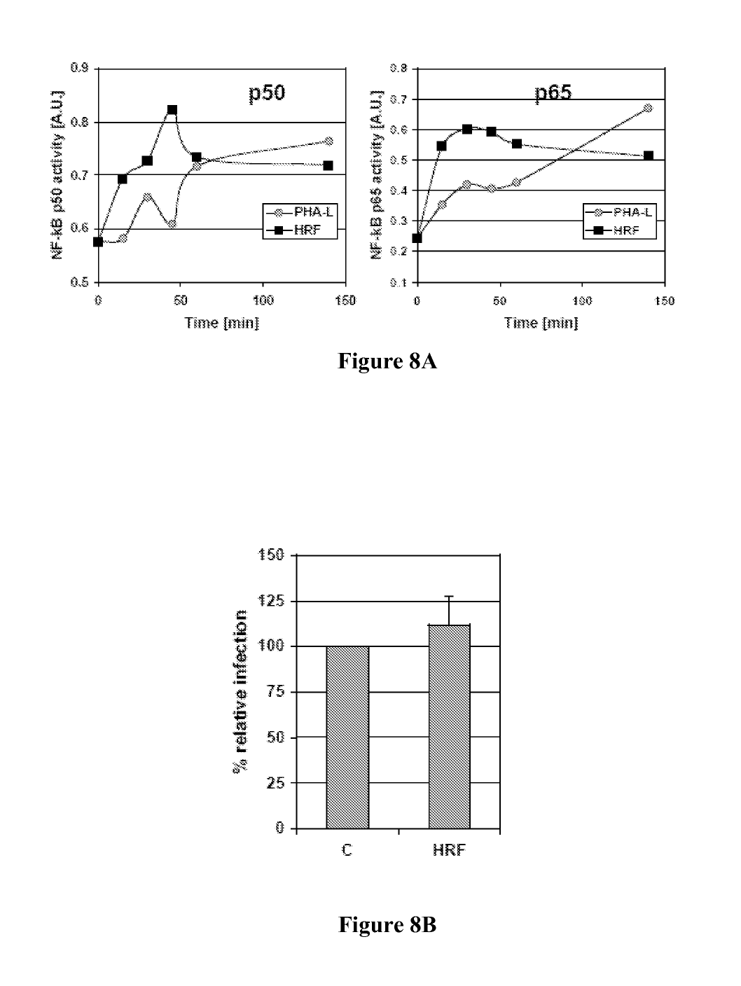

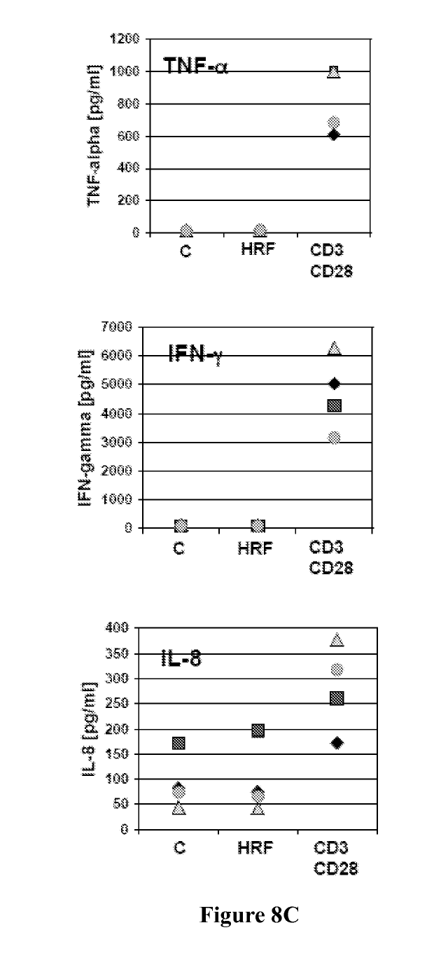

One of the crucial problems with any stimulatory approach aimed at reactivation of latent HIV-1 infection in PBMCs is the question of whether HIV-1 activation can be dissociated from the induction of cytokine expression that would potentially lead to a hypercytokinemia. To test this, it was initially determined that HRF stimulation induced the same high peak of NF-κB activity observed in Jurkat cells. Indeed, when compared to PHA-L, a plant lectin that is commonly used to activate primary T cell cultures, HRF induced a very high, but short-lived NF-κB p50 and p65 activity peak (FIG. 8A) that was comparable to that seen in Jurkat cells (FIG. 7). HIV-1 replication in PBMCs in the presence of saturating HRF levels was not significantly increased in an experiment tracing HIV-1 replication levels for 5 days post infection (FIG. 8B). However, while HRF stimulation of PBMCs induced NF-κB activity, HRF did not induce meaningful levels of pro-inflammatory cytokine secretion (IL-8, TNF-α, and IFN-γ) (FIG. 8C). These data confirm the results using promoter constructs (FIG. 6C) and suggest that HRF stimulation results only in minimal cellular promoter activation.

### Example 2

### Identification of HIV-1 Reactivating Drug Combinations: Dactinomycin Primes Latent HIV-1 Infection for Reactivation

### Materials and Methods

Cell Culture, Plasmids and Reagents.

All T cell lines were maintained in RPMI 1640 supplemented with 2 mM L-glutamine, 100 U/ml penicillin, 100 μg/ml streptomycin and 10% heat inactivated fetal bovine serum. The latently infected J89GFP cells, CA5 T cells, and EF7 T cells have been described earlier (Duverger et al., J. Virol. 83:3078-93 (2009)). Fetal bovine serum (FBS) was obtained from HyClone (Logan, Utah) and was tested on a panel of latently infected cells to assure that the utilized FBS batch did not spontaneously trigger HIV-1 reactivation (Duverger et al., J. Virol. 83:3078-93 (2009); Kutsch et al., J. Virol. 76:8776-86 (2002)). The phorbol ester 13-phorbol-12-myristate acetate (PMA), rabaccamycin and oxaliplatin were purchased from Sigma (St. Louis, Mo.), whereas recombinant human TNF-α was obtained from R&D Systems (Minneapolis, Minn.). Daunorubicin, α-amanitin, IRCF-193 and camptothecin were purchased from Calbiochem (EMD Chemicals; Gibbstown, N.J.). 5,6 dichloro-beta-Dribofuranosylbenzimidazole (DRB) was purchased from ALEXIS Biochemicals (San Diego, Calif.). A retroviral MSCV-DsRedExpress plasmid was used for generation of the RFP-barcoded J89GFP cell populations.

J2574 Reporter T Cells.

J2574 reporter T cells were generated by retrovirally transducing Jurkat T cells with a HIV-1 reporter construct (p2574) in which the HIV-1 LTR controls the expression of GFP. The HIV-1 LTR and the GFP gene are separated by a 2,500 base pair (bp) spacer element. Lentiviral particles were produced by transfecting 293T cells with p2574 and supplying gag-pol-rev-tat in trans. VSV-G was used as viral envelope protein. Following lentiviral transduction of Jurkat cells, all cells that spontaneously expressed GFP were removed by cell sorting. The GFP-negative population was then activated with PMA to identify all cells that would harbor an inducible LTR-GFP-LTR integration event. Cells that turned GFP-positive following stimulation were again selected by cell sorting. GFP expression in this population ceased after a few days leaving a population of GFP-negative reporter cells. The amount of founder cells for this population is calculated to represent >50,000 individual integration events.

Glycerol Gradient Sedimentation Analysis.

Ten million J89GFP or CA5 T cells were left untreated or treated with 0.004 μg/mL, 0.01 μg/ml or 1 μg/mL dactinomycin for 18 hours or 1 hour, respectively. Cells were washed twice with cold PBS, then lysed for 30 minutes on ice in lysis buffer (0.5% TritonX100, 20 mM HEPES (pH7.9), 150 mM NaCl, 20 mM KCl, 2 mM MgCl2, 1 mM DTT, 0.2 mM EDTA, and protease inhibitor cocktail (P8340; SIGMA)), followed by centrifugation at 14,000 rpm for 10 minutes. The same amount of protein lysate was fractionated on 5 ml of a 10-45% glycerol gradient in lysis buffer in a SW-Ti55 rotor (Beckman Coulter; Miami, Fla.) for 16 hours at 45,000 rpm. Fractions were resolved on 10% SDS-PAGE and transferred to polyvinylidene fluoride membrane. The antibodies used for Western blotting were rabbit anti-Cdk9 (sc-484; Santa Cruz Biotechnology; Santa Cruz, Calif.) and rabbit anti-HEXIM1 (ab25388; Abcam; Cambridge, Mass.), respectively.

Flow Cytometry.

Infection levels in the cell cultures were monitored by flow cytometric (FCM) analysis of GFP expression. FCM analysis was performed on a GUAVA EasyCyte (GUAVA Technologies, Inc.; Millipore; Billerica, Mass.) and a BD FACSCalibur or a LSR11 (Becton & Dickinson; Franklin Lakes, N.J.). Cell sorting experiments were performed using a FACSAria™ Flow Cytometer (Becton&Dickinson). Data analysis was performed using either CellQuest (Becton&Dickinson) or GUAVA Express (GUAVA Technologies, Inc.).

High Throughput Drug Screening.

HTS data acquisition was performed as described in FIG. 1 using a HyperCyt autosampler combined with a FACSCalibur flow cytometer. The system was adjusted to acquire 2,000 counts in the life gate to ensure sufficiently high cell counts to perform statistically meaningful data analysis. The assay is characterized by a Z′-factor of 0.83 using PMA as an activating agent. Maximum achievable HIV-1 reactivation levels for the three populations using 10 ng/ml PMA were 90±3%. Data analysis was performed using the HyperView® Data Analysis Software (Intellicyt; Albuquerque, N. Mex.). Determination of hits can be visually performed using a heat-map that is programmed to indicate changes in HIV-1 expression levels by a self-defined color code. HyperView-generated data were transferred to Spotfire (TIBCO; Somerville, Mass.) or Excel (Microsoft; Redmond, Wash.) for statistical analysis.

Compound plates for drug screening purposes were generated from a parental 80,000 compound library (ChemBridge; San Diego, Calif.) using a BioTek Precision platform (BioTek, Winooski, Vt.). In addition, an in-house collection of drugs/compounds with known molecular function was utilized.

### Results

Drug Screening Assay.

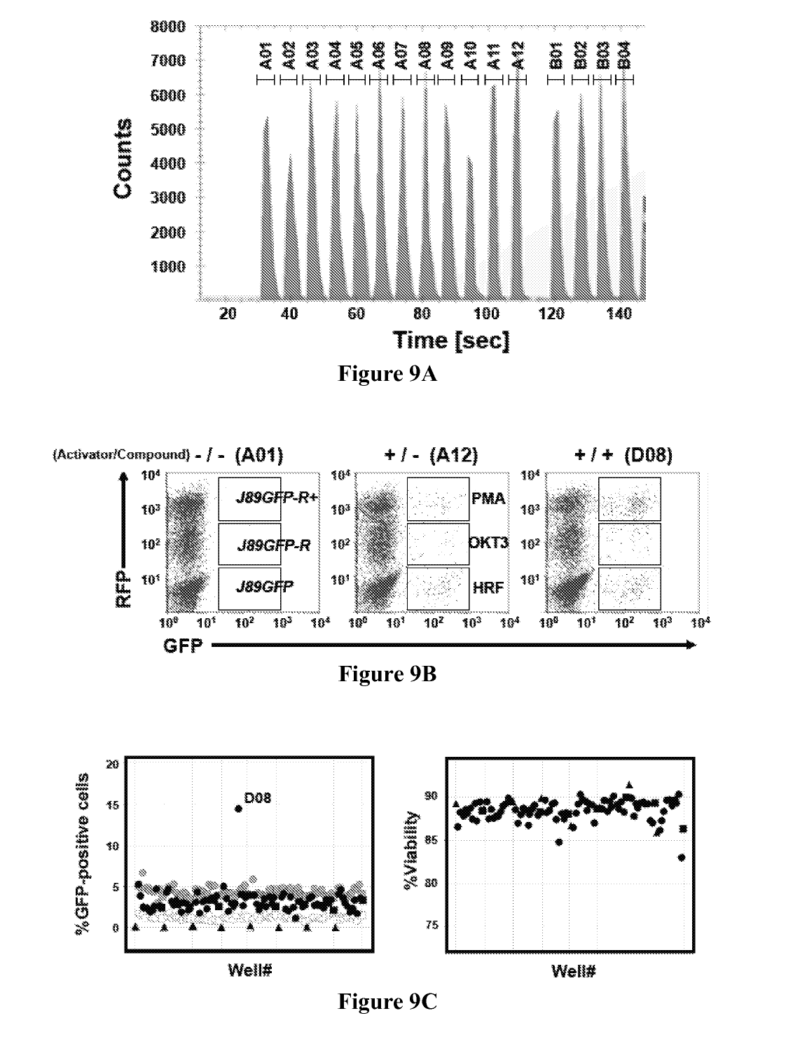

A high quality high throughput drug screen (HTS) condenses the key elements that define the therapeutic target in vivo into a 96-well or higher plate-based assay format. In this case, a HTS was developed to directly identify drug combinations with superior HIV-1 reactivating capacity relative to single compounds. The drug combination to be identified was aimed to consist of a modulator compound and a mild activator. To detect even weak hits, flow cytometry was chosen as the most sensitive read-out available and the assay was based on the previously reported latently HIV-1 infected J89GFP T cells (Kutsch et al., J. Virol. 76:8776-86 (2002)). J89GFP cells were latently infected with a GFP reporter virus. In a latent state, the cells do not express GFP; however, following reactivation by stimuli such as anti-CD3/CD28 mAb combinations, TNF-α or PMA, the cells start to express high levels of GFP as a direct and quantitative marker of HIV-1 expression. With GFP being used as the specific signal for on-target drug effects, J89GFP cells were transduced with a retroviral DsRedExpress (RFP) vector to produce three distinctive J89GFP populations (J89GFP, J89GFP-R, J89GFP-R++), distinguishable by a RFP-based fluorescent barcode (FIG. 9B). Retroviral transduction was performed using a MSCV-LTR based retroviral vector to express RFP, as MSCV-LTR-driven gene expression in Jurkat T cells remains stable in long-term cell culture and does not respond to activation with changes in fluorescence intensity. The latter characteristic maintained the integrity of the fluorescent barcode following compound addition.

Screen for Modulator Compounds.

In a limited screening effort designed to define the quality of the HTS assay, a 2,000 compound library holding an extensive selection of drugs/compounds with defined activities was used. The drug screen was designed to identify modulator compounds that were able to prime latent HIV-1 infection for reactivation by sub-threshold concentrations of three predetermined activators (PMA, OKT3, and HRF (Wolschendorf et al., J. Virol. 84(17):8712-20 (2010)) in a single 96-well plate. Final compound concentrations were chosen at 5 μM for compounds derived from our 80,000 small chemical molecule library (Chembridge). Compound concentrations in the in-house library varied according to the known effective concentrations of the respective compounds.

For the modulator compound screen, three individual 96-well plates holding either 1×10⁵/well J89GFP, J89GFP-R++ and J89GFP-R++ cells were prepared. Compounds were loaded into the individual wells, and after 6 hours, the individual plates were stimulated with either sub-optimal concentrations of PMA, OKT3 or HIV-1 Reactivating Factor (HRF) as activators. Each activator concentration was adjusted to have minimal or no HIV-1 reactivating effect by itself. Twenty-four hours after addition of the compounds, the 3 corresponding individual 96-well plates were combined using a robotic platform. The plates were immediately subjected to high-throughput flow cytometric analysis using a HyperCyt® high throughput autosampler, which allows for time-resolved data acquisition (FIG. 9A). In this setup, the data for individual samples were not collected as single files, but as time-resolved data. Separation of the individual data sets was achieved using a specialized analysis software (HyperView® Data Analysis Software). As a function of the cell density of a sample and the required amount of events, the technology allows for extremely fast immediate multi-parameter analysis. The established RFP barcode subsequently allowed testing of several drug combinations per well in one single analytical run using a HyperCyt autosampler in conjunction with a FACSCalibur flow cytometer to achieve high throughput. In the experimental set-up, by combining HTS flow cytometry with a fluorescent barcode (FIG. 9B), a 96-well plate or 288 drug combinations were analyzed in 8 minutes when 2×10³ cells per population were acquired. The following parameters were determined in each plate during the primary drug screen: HIV-1 transcription activity for each cell population, compound-induced changes in the base-line GFP expression, cell density of each cell population as characterized by individual fluorescence signatures (anti-proliferative compound effects), and overall cell viability as determined by life gate analysis in the FSC/SSC plot (compound toxicity). The results of a 96-well sample plate are shown in FIG. 9C. One major advantage of flow cytometry-based drug screening in this system is that on-target effects are sensitively detected despite significant compound toxicities at the utilized compound concentration. As hits can be identified by determining the ratio of cells harboring latent (GFP-negative) to active infection (GFP-positive) within one population determined by the RFP barcode, the assay became largely independent of the amount analyzed cells. On-target effects were still detected in the presence of massive compound toxicity.

Dactinomycin Primes Latent HIV-1 Infection for Efficient Reactivation.

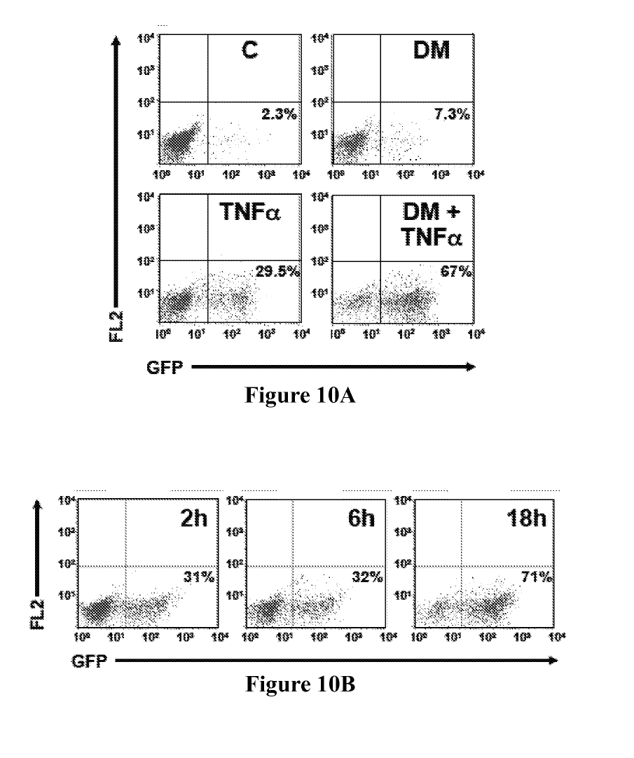

During the initial limited 2,000 compound screen, a total of 13 modulator compounds were identified. The modulating activity of 80% of these compounds was confirmed in verification assays. Interestingly, compounds identified in previous drug screens that directly triggered HIV-1 reactivation almost exclusively exerted their activity at concentrations associated with the onset of cytotoxic side effects. Surprisingly, several of the compounds identified in this drug screen exerted their priming activity in the absence of any cytotoxic side effects. As HIV-1 latency does not offer a defined molecular target and the drug screen was based on a change in phenotype, the mechanism of action for each identified hit had to be determined individually. Described herein, one of the identified hits with potent modulator activity, dactinomycin, exerted its priming effect on latent HIV-1 infection (FIG. 10). Relative to the latently infected control cells, dactinomycin did not exhibit HIV-1 reactivating capacity by itself. However, in combination with sub-optimal concentrations of an activator (here TNF-α), it potently primed latent HIV-1 infection for reactivation. The optimal duration of a pretreatment period for dactinomycin prior to addition of the activating stimulus was determined. For this purpose, dactinomycin was added for 2, 6 or 18 hours to the latently HIV-1 infected and J89GFP T cells. The cells were then stimulated with a sub-optimal concentration of TNF-α. The experiments revealed that the optimal pretreatment time is 18 hours prior to the addition of the reactivating stimulus.

To identify the optimal concentration of dactinomycin, CA5 T cells were pretreated with increasing concentrations of dactinomycin (0.0001 and 1 μg/ml), and then left untreated or stimulated with a sub-optimal concentration of HRF or TNF-α. Representative results for stimulation with HRF are depicted in FIG. 3. The chosen HRF concentration has only minimal HIV-1 reactivating effect by itself (15% reactivation over background). Twenty four hours after activator addition, levels of HIV-1 reactivation were determined as the percentage of GFP-positive cells using flow cytometric analysis, which allowed for the simultaneous determination of cell viability. The experiments revealed that, in both T cell lines, dactinomycin exerted its optimal priming activity for latent HIV-1 infection at a concentration of about 4 ng/ml (3.18 nM), with a priming effect being observed at concentrations as low as 0.5 ng/ml. Optimal pretreatment time was 18 hours. Maximum reactivation effects were seen 48 hours after stimulation. Priming effects of dactinomycin were also observed when low concentrations of TNF-α or PMA were used as reactivating agents.

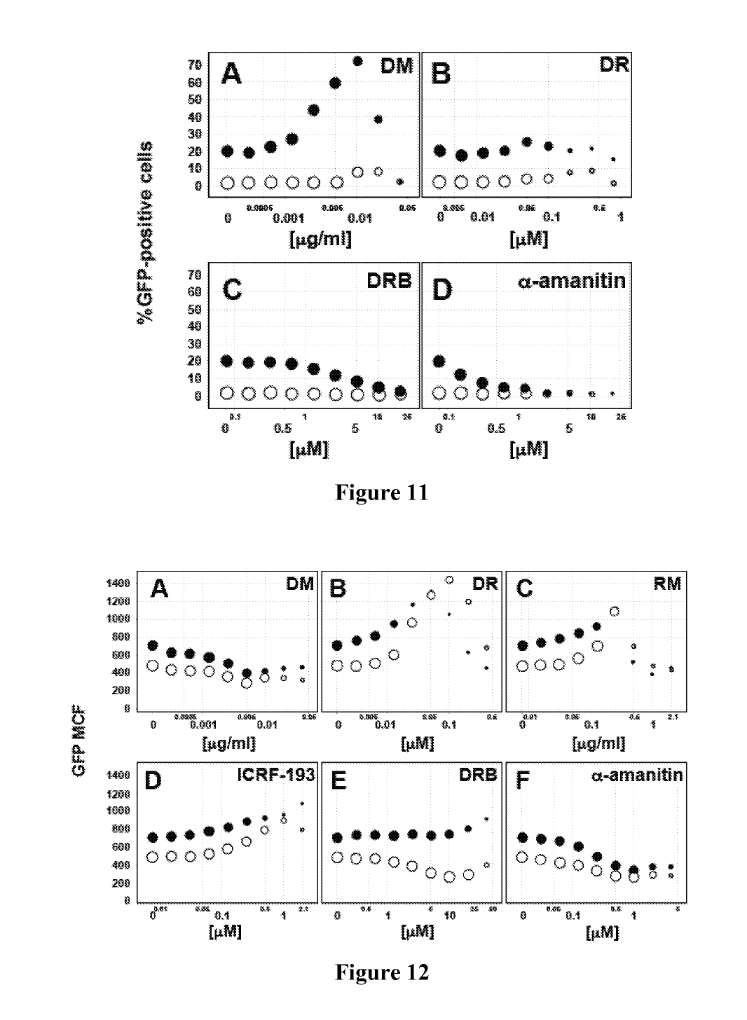

One function by which dactinomycin exerts its on-target drug effect is DNA intercalation. To this end, the effect of other DNA intercalators, such as daunorubicin, rebeccamycin, oxaliplatin or amsacrine, on latent HIV-1 infection was tested to determine whether the priming effect for latent HIV-1 infection exerted by dactinomycin was related to its ability to act as a DNA intercalator. The DNA intercalators were titrated on CA5 T cells and incubated for various amounts of time prior to triggering reactivation by a sub-optimal dose of HRF. The experiments revealed that the effect was specific for dactinomycin and was not reproduced by the other tested DNA intercalators (data for daunorubicin with 18 hour pretreatment shown in FIG. 11B). The data do not suggest that DNA intercalation is the primary mode of action required by dactinomycin to prime latent HIV-1 for reactivation.

A second reported inhibitory function of dactinomycin is its ability to block RNAP II, an activity that is likely related to its ability to intercalate into DNA. Thus, the transcription inhibitors α-amanitin, IRCF-193, camptothecin, or 5,6 dichloro-beta-D-ribofuranosylbenzimidazole (DRB) were tested for their ability to prime latent HIV-1 infection for reactivation. Again to ensure that potential compound effects were not missed because of ineffective pre-treatment times, all experiments were performed using 2 hour and 18 hour pretreatment periods. None of the inhibitors exerted a priming effect on latent HIV-1 infection. Data for DRB and α-amanitin are shown in FIGS. 11C and 11D. As expected for transcription inhibitors, higher concentrations of either drug were found to inhibit HIV-1 reactivation triggered by HRF or TNF-α, and this inhibitory activity was correlated with high levels of cytotoxicity, likely triggered by the general inhibition of the cellular transcription machinery.

It is noteworthy that dactinomycin at higher concentrations (e.g., concentrations great than or equal to 10 ng/ml) also starts to act as an inhibitor of HIV-1 expression and reactivation, which is consistent with its function as a RNAP II inhibitor. This effect is observed at the onset of drug toxicities, suggesting that these dactinomycin concentrations also start to affect general transcription. (FIG. 11A)

Influence of Dactinomycin on Active HIV-1 Infection.

Eradication of HIV-1 reservoirs by reactivating latent HIV-1 infection events will have to be achieved under treatment conditions that would prevent all de novo infection. There is a high likelihood that this can be achieved by intensifying standard ART with entry or integration inhibitors during the application of HIV-1 reactivating drugs. Nevertheless, it is likely advantageous to develop HIV-1 reactivating drugs that do not boost active HIV-1 infection, to minimize the risk of de novo infection. The effect of dactinomycin and a series of other DNA intercalators (daunorubicin, rebeccamycin) and transcription inhibitors (ICRF-193, DRB, α-amanitin) were tested on active HIV-1 infection to assure that no priming effect is seen on active infection (FIG. 12). For this purpose, each of the compounds was titrated over a relevant range of concentrations on two chronically actively infected T cell lines (JNLG#35 and JNLG#44). These clonal cell lines are infected with a GFP-reporter virus. Forty eight hours after addition of the compounds, the effect of each compound on HIV-1 transcription was determined by flow cytometric analysis quantifying GFP mean channel fluorescence intensity (MCF). Dactinomycin, at concentrations relevant for HIV-1 reactivation, did not boost active infection, but rather inhibited active infection (FIG. 12A). Interestingly, some agents that did not trigger HIV-1 reactivation boosted active HIV-1 transcription, such as the DNA intercalators daunorubicin or rebeccamycin (FIGS. 12B and 12C). This is likely the result of the reported ability to stimulate NF-κB activity. In neither case the effects of the tested transcription inhibitors were pronounced in the absence of cytotoxic effects.

In summary, these data demonstrated that dactinomycin achieved its priming effect for HIV-1 reactivation without boosting active HIV-1 infection. As there is no indication that the proposed primary effect of dactinomycin was as a DNA intercalator or as a transcription inhibitor with the observed effect on active HIV-1 expression, these data further suggested that the priming effect of dactinomycin was achieved by a different mechanism of action.

Potential Influence of Dactinomycin on Transcriptional Interference Effects Controlling Latent HIV-1 Infection.

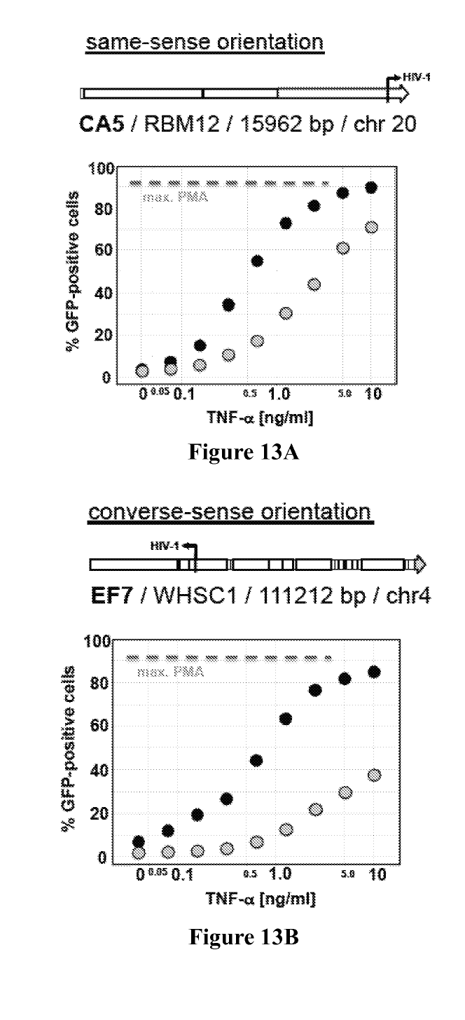

The sense of orientation of the integrated latent virus, relative to the transcriptional direction of the host-gene, was investigated to determine whether the orientation would influence the ability of dactinomycin to prime latent infection for reactivation. In the latently HIV-1 infected CA5 T cells, virus and host-gene were oriented in the same transcriptional orientation, whereas in EF7 cells, the virus was integrated into the host-gene in the converse transcriptional orientation (FIGS. 13A and 13B). As shown in FIG. 13, dactinomycin exerted its priming effect in each integration scenario, suggesting that the priming effect is unlikely to be caused by transcriptional interference effects that may add to the control of latent infection. The data rather suggested that dactinomycin treatment favored direct LTR activation or promoted elongation efficacy.

Dactinomycin Exerts Priming Activity on Latent HIV-2 Infection.

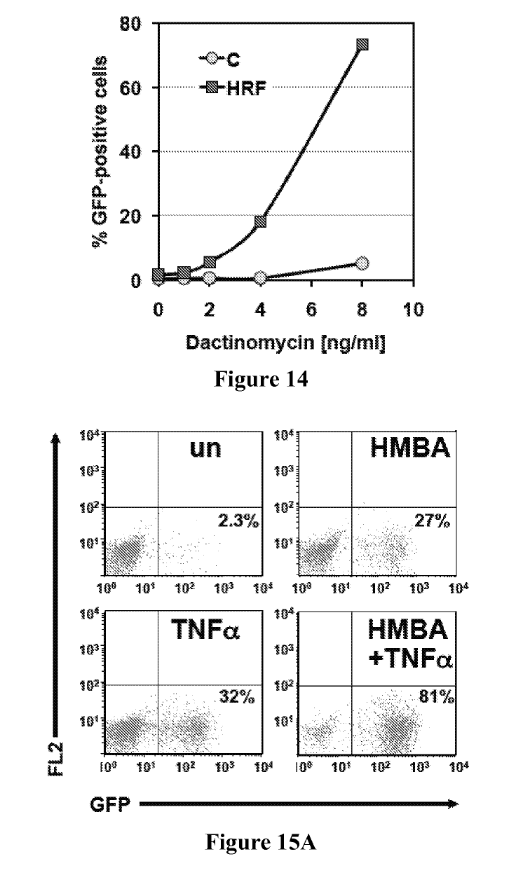

Next, the observed priming effect for latent HIV-1 infection was investigated to determine if the priming effect was specific for HIV-1 or if there was a priming effect for latent HIV-2 infection. To determine the ability of dactinomycin to prime a latent HIV-2 infection, a latently HIV-2 infected population of GFP reporter T cells were tested. Briefly, to create the latent HIV-2 infected population, J2574 reporter cells were infected with HIV-2 7312A and a population of latently HIV-2 infected cells was generated by removing the actively infected, GFP-positive cells using a fluorescence activated cell sorter. In the remaining GFP-negative population, >90% of the cells were latently infected as revealed by PMA stimulation. Dactinomycin efficiently primed latent HIV-2 infection for reactivation in a range between 2-8 ng/ml (FIG. 14). Background active infection in the control population was 1% and addition of a sub-optimal dose of HRF resulted in HIV-2 reactivation in 10% of the cells. Costimulation of the latently HIV-2 infected cell population resulted in reactivation levels of up to 90%. These data showed that dactinomycin exerted its priming activity on a component of the transcriptional control shared by both, HIV-1 and HIV-2.

Dactinomycin Releases P-TEFb from the Inactive Complex with HEXIM-1.

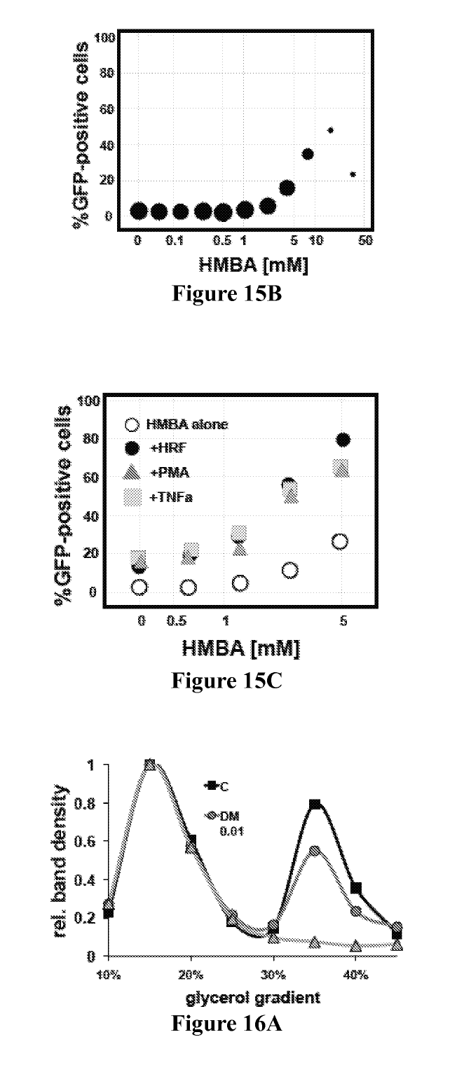

As the data indicated that the priming effect of dactinomycin on latent HIV-1 infection was triggered at the level of transcriptional elongation, the possibility that dactinomycin released positive transcription elongation factor (P-TEFb) from its inactive complex with HEXIM-1 was investigated. P-TEFb-association to RNAP II is essential to trigger efficient elongation and the presence of P-TEFb (a complex of cyclin T1 and CDK9) at the RNAP II complex associated with the HIV-1 LTR has been demonstrated as essential for efficient transcriptional elongation. Hexamethylene bisacetamide (HMBA) mediated release of P-TEFb from its complex with HEXIM-1 triggers HIV-1 reactivation. HMBA triggered some level of HIV-1 reactivation in the latently HIV-1 infected CA5 T cells, however, reactivation levels were low (<40%) when compared to activators such as TNF-α, PMA or HRF. At 3 mM, HIV-1 was reactivated in 15% of the cells and at 9 mM reactivation was triggered in 35% of the cells; however, at this concentration, reactivation correlated with the onset of compound toxicity. Nevertheless, HMBA at sub-toxic concentrations was relatively potent at priming latent HIV-1 infection for full reactivation by a sub-optimal activating TNF-α concentration (FIG. 15).

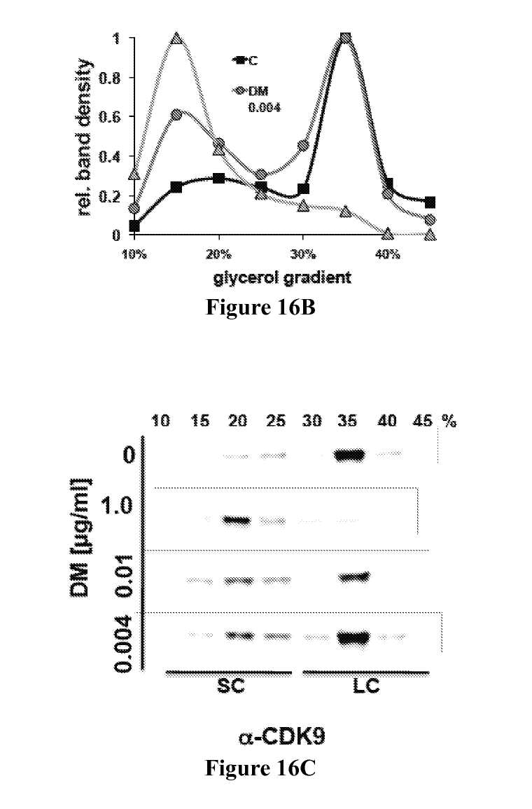

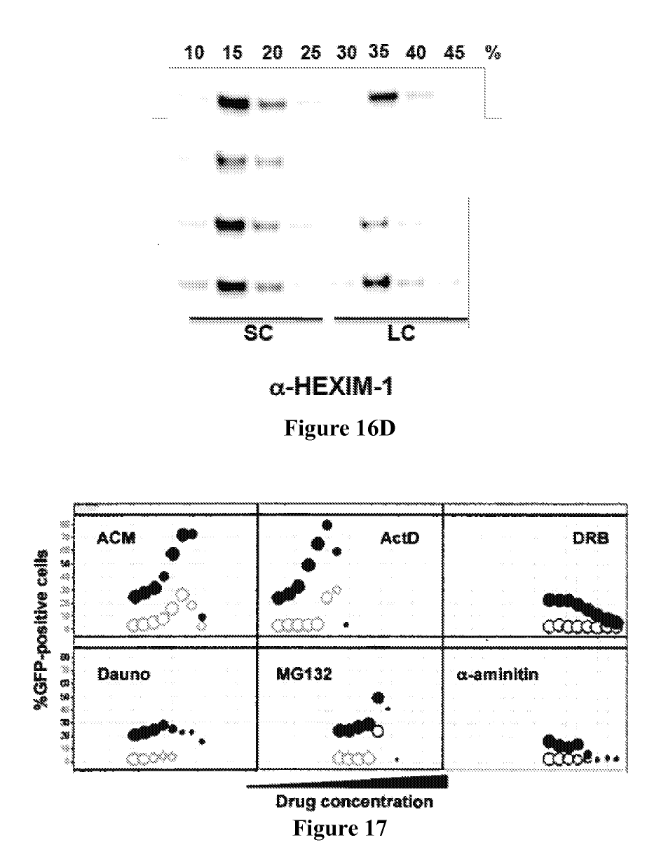

To test the idea that dactinomycin primed latent HIV-1 infection for reactivation by releasing P-TEFb (a complex of CDK9 and Cyclin T1) from its complex with HEXIM-1, the latently HIV-1 infected J89GFP or CA5 T cells were treated with 1.0 μg/ml dactinomycin for 1 hour or with the physiological optimal concentration of 0.004n/ml for 18 hours. Cell lysates were then separated on a glycerol gradient (10-45%) to reveal possible changes in the composition of the P-TEFb/HEXIM-1 complex. Release of P-TEFb from the inactive complex with HEXIM-1 (large complex), which is found in the glycerol gradient fractions with higher glycerol content, was indicated by a shift to a smaller complex (CDK9/CycT1) found in the gradient fractions with lower glycerol content. Each gradient fraction was separated on a SDS-PAGE gel and subjected to Western blotting. The results of these experiments using J89GFP cells are presented in FIG. 16. Staining with anti-CDK9 antibody revealed that treatment of J89GFP with 1 μg/ml dactinomycin for 1 hour quantitatively released P-TEFb from its complex with HEXIM-1. A shift of CDK9 presence from the large complex to the small complex was also detected under treatment conditions that represented the optimal conditions for HIV-1 reactivation (0.004 μg/ml dactinomycin for 18 hours). Similar results were obtained using anti-HEXIM-1 antibody. However, for HEXIM-1, no shift towards the small complex was observed at the optimal condition of 0.004 μg/ml dactinomycin for 18 hours. The minimal dactinomycin concentration to induce a shift towards the small complex was 0.01 μg/ml of dactinomycin. Other than CDK9, HEXIM-1, even in control cells, was found in the small complex fractions, suggesting that free HEXIM-1 is present in abundance, which was consistent with the idea that it served as a regulator of transcription by inactivating P-TEFb. Similar results were obtained using the latently infected CA5 T cells. In summary, the experiments suggested that the priming effect of dactinomycin was induced by the release of P-TEFb from its inactive complex with HEXIM-1, which favor elongation of transcription by the paused RNAP II complex found at the latent HIV-1 LTR.

1. A method of reactivating a latent Human Immunodeficiency Virus (HIV) infection in a cell comprising modulating a level of NF-κB activity in the cell by contacting the cell with a first agent that produces a transient first increase in the level of NF-κB activity without a second delayed increase in NF-κB activity.

2. The method of claim 1, wherein the second delayed increase in NF-κB activity is associated with cytokine gene induction, wherein the absence of a second delayed increase in NF-κB activity is accompanied by an absence of cytokine gene induction.

3. The method of claim 1, wherein the modulation in NF-κB activity differs in pattern from a modulation caused by TNF-α, PMA, PHA-L, IL-2, anti-CD3 monoclonal antibodies, or a combination of anti-CD3 and anti-CD28 monoclonal antibodies.

4. The method of claim 1, wherein the modulation in the level of NF-κB activity is detected as a modulation in the level of NF-κB p50 activity or as a modulation of the level of NF-κB p65 activity.

5. (canceled)

6. The method of claim 2, wherein the absence of gene induction comprises the absence of induction of one or more of TNF-α, IL-8, IFNγ, IL-2, IL-4, and IL-6.

7. The method of claim 1, wherein the modulation in the level of NF-κB activity is not accompanied by the induction of HIV replication.

8. (canceled)

9. (canceled)

10. The method of claim 1, wherein the method further comprises contacting the cell with a second agent that primes the latent HIV infection.

11. (canceled)

12. (canceled)

13. The method of claim 10, wherein the second agent is selected from the group consisting of actinomycin D, aclacinomycin, amphotericin B, and WP631.

14. (canceled)

15. The method of claim 14, wherein the actinomycin D is administered at a dose of 15 micrograms per kilogram per day (μg/kg/day).

16. An isolated Massilia bacterium or population thereof for producing a Human Immunodeficiency Virus (HIV) reactivating factor (HRF).

17. An isolated Human Immunodeficiency Virus (HIV) reactivating factor (HRF) produced by the Massilia bacterium of claim 16.

18. The Massilia bacteria of claim 16, wherein the Massilia bacteria comprises a 16S rRNA sequence, wherein the 16S rRNA sequence comprises at least 95% sequence identity with Massilia timonae.

19. (canceled)

20. A composition comprising a purified population of a Massilia timonae strain having ATCC Accession number PTA-10969.

21. An isolated Human Immunodeficiency Virus (HIV) reactivating factor (HRF) produced by the Massilia strain of claim 20.

22. (canceled)

23. (canceled)

24. An isolated Human Immunodeficiency Virus (HIV) reactivating factor (HRF) produced by the Massilia timonae strain having ATCC accession number BAA-703.

25. A method of reactivating a latent Human Immunodeficiency Virus (HIV) infection in a subject comprising administering to a subject a HIV reactivating factor (HRF) produced by Massilia bacteria or a reactivating fragment of the HRF produced by Massilia bacteria.

26. The method of claim 25, wherein the HRF is produced by a Massilia timonae strain having ATCC Accession number PTA-10969.

27. The method of claim 25, wherein the HRF is produced by Massilia timonae strain having ATCC accession number BAA-703.

28. (canceled)

29. (canceled)

30. The method of claim 25, wherein the method further comprises administering to the subject an agent that primes the latent HIV infection.

31. (canceled)

32. (canceled)

33. The method of claim 30, wherein the agent is selected from the group consisting of actinomycin D, aclacinomycin, amphotericin B, and WP631.

34. (canceled)

35. A method of treating an HIV infection in a subject, the method comprising:
(a) administering to the subject a first agent that reactivates a latent HIV infection by modulating a level of NF-κB activity, wherein modulation of the level of NF-κB activity comprises producing a transient first increase in the level of NF-κB activity without a second delayed increase in NF-κB activity; and
(b) administering to the subject an anti-retroviral agent, wherein administration to the subject of the anti-retroviral agent treats the HIV infection.

36. The method of claim 35, wherein the anti-retroviral agent is administered to the subject after reactivation of the latent HIV infection.

37. (canceled)

38. The method of claim 35, wherein the method further comprises administering to the subject a second agent that primes the latent HIV infection in the subject.

39. The method of claim 38, wherein the second agent is administered prior to the first agent.

40. (canceled)

41. The method of claim 38, wherein the second agent is selected from the group consisting of actinomycin D, aclacinomycin, amphotericin B, and WP631.

42. (canceled)

43. The method of claim 35, wherein the first agent is an HIV reactivating factor (HRF) or a reactivating fragment thereof produced by Massilia bacteria.

44. The method of claim 43, wherein the Massilia bacteria is a Massilia timonae strain having ATCC Accession number PTA-10969.

45. A method of producing an HIV reactivating factor comprising (a) culturing a Massilia bacteria in a mammalian cell culture medium under conditions that allow for the secretion of the HRF into the culture media; and (b) isolation of the Massilia bacterial conditioned media.

46. The method of claim 45, wherein the mammalian cell culture medium is a RPMI 1640 medium.

47. The method of claim 46, wherein the RPMI 1640 medium further comprises a mammalian serum, bovine serum albumin (BSA), or myoglobin.

48-52. (canceled)


SEQUENCE LISTING (8 sequences)

SEQ ID NO: 1 - RNA, 1398 nt, Massilia timonae
  16s rRNA sequence
ugaacgcuggcggcaugcuuuacacaugcaagucgaacgguaacagggagcuugcuccgcugacgaguggcgaacgggugaguaauauaucggaacguacccaagagugggggauaacguagcgaaaguuacgcuaauaccgcauacgaucuauggaugaaagcaggggaccuucgggccuugugcuccuggagcggccgauaucugauuagcuaguuggugagguaaaggcucaccaaggcgacgaucaguagcuggucugagaggacgaccagccacacuggaacugagacacgguccagacuccuacgggaggcagcaguggggaauuuuggacaaugggcgcaagccugauccagcaaugccgcgugagugaagaaggccuucggguuguaaagcucuuuugucagggaagaaacggcuguggcuaauauccacggcuaaugacgguaccugaagaauaagcaccggcuaacuacgugccagcagccgcgguaauacguagggugcaagcguuaaucggaauuacugggcguaaagcgugcgcaggcgguuuuguaagucugucgugaaauccccgggcucaaccugggaauugcgauggagacugcaaggcuagaaucuggcagagggggguagaauuccacguguagcagugaaaugcguagagauguggaggaacaccgauggcgaaggcagcccccugggucaagauugacgcucaugcacgaaagcguggggagcaaacaggauuagauacccugguaguccacgcccuaaacgaugucuacuaguugucggguuuuaauuaacuugguaacgcagcuaacgcgugaaguagaccgccuggggaguacggucgcaagauuaaaacucaaaggaauugacggggacccgcacaagcgguggaugauguggauuaauucgaugcaacgcgaaaaaccuuaccuacccuugacaugucaggaagrccgaagagauuugguugugcccgaaagggaaccugaacacaggugcugcauggcugucgucagcucgugucgugagauguuggguuaagucccgcaacgagcgcaacccuugucauuaguugcuacgcaagagcacucuaaugagacugccggugacaaaccggaggaagguggggaugacgucaaguccucauggcccuuauggguagggcuucacacgucauacaaugguacauacagagggccgccaacccgcgagggggagcuaaucccagaaaguguaucguaguccggaucguagucugcaacucgacuacgugaaguuggaaucgcuaguaaucgcggaucagcaugccgcggugaauacguucccgggucuuguacacaccgcccgucacaccaugggagcggguuuuaccagaaguag

SEQ ID NO: 2 - DNA, 33 bp, Artificial Sequence
  Synthetic Construct
ggcgcggagatcttaacgaagacagggccatgt

SEQ ID NO: 3 - DNA, 32 bp, Artificial Sequence
  Synthetic Construct
gccaataccggtgtgtcctttccaggggagag

SEQ ID NO: 4 - DNA, 20 bp, Artificial Sequence
  Synthetic Construct
agagtttgatcctggctcag

SEQ ID NO: 5 - DNA, 21 bp, Artificial Sequence
  Synthetic Construct
acggctaccttgttacgactt

SEQ ID NO: 6 - DNA, 24 bp, Artificial Sequence
  Synthetic Construct
ccctaaacgatgtctactagttgt

SEQ ID NO: 7 - DNA, 24 bp, Artificial Sequence
  Synthetic Construct
ttcgggcacaaccaaatctcttcg

SEQ ID NO: 8 - DNA, 23 bp, Artificial Sequence
  Synthetic Construct
ggctcaacctcccaattgcgatg